(19) United States
(12) Patent Application Publication
(10) Pub. No.: US 2011/0207804 A1
(43) Pub. Date: Aug. 25, 2011

(54) COMPOSITIONS FOR THE TREATMENT OF NEOPLASTIC DISEASES

(75) Inventors: Jacob Hendrik Beijnen, Amsterdam (NL); Johannes Henricus Matthias Schellens, Amsterdam (NL); Johannes Moes, Amsterdam (NL); Bastiaan Nuijen, Amsterdam (NL)

(73) Assignees: Slotervaart Participaties BV, Amsterdam (NL); Stichting Het Nederlands Kanker Institut, Amsterdam (NL)

(21) Appl. No.: 13/060,037

(22) PCT Filed: Aug. 24, 2009
(86) PCT No.: PCT/GB2009/002068

§ 371 Date: May 11, 2011

(30) Foreign Application Priority Data
Aug. 22, 2008 (GB) PCT/GB2008/002854

(51) Int. Cl. A61K 31/337; A61P 35/00

(52) U.S. Cl. 514/449

(57) ABSTRACT

Solid pharmaceutical taxane compositions for oral administration which comprise a substantially amorphous taxane, a carrier, and a surfactant, wherein the substantially amorphous taxane is prepared by a solvent evaporation method, such as spray drying. Methods of preparation of the composition and uses of the composition also are included.

The invention relates to pharmaceutical compositions. In particular, though not exclusively, it relates to compositions for the treatment of neoplastic disease.

The administration of drugs in oral form provides a number of advantages. The availability of an oral anticancer drug is important when treatment must be applied chronically to be optimally effective e.g., the 5-fluorouracil (5-FU) prodrugs (e.g. capecitabine) and drugs that interfere with signal transduction pathways or with the angiogenesis process [1]. In addition, oral drugs can be administered on an outpatient basis or at home, increasing convenience and patient quality of life, and possibly decreasing costs by reducing hospital admissions [2]. Therefore, it is advantageous to try to administer anticancer drugs orally.

In general, the oral administration of drugs is convenient and practical. However, the majority of anticancer drugs unfortunately have a low and variable oral bioavailability [1]. Typical examples are the widely used taxanes, docetaxel and paclitaxel, which have an oral bioavailability of less than 10% [3, 4]. Several other anticancer agents with higher bioavailability demonstrate higher variability. Examples include the topoisomerase I inhibitors, the vinca alkaloids, and mitoxantrone [1, 5, 6]. In view of the narrow therapeutic window, the variable bioavailability may result in unanticipated toxicity or decreased efficacy when therapeutic plasma levels are not achieved. Hellriegel et al. demonstrated in a study that the plasma levels after oral administration are generally more variable than after i.v. administration [7]. Adequate oral bioavailability is important when the period of drug exposure is a major determinant of anticancer therapy [8]. Adequate oral bioavailability is also important to prevent high local drug concentrations in the gastro-intestinal tract that may give local toxicity.

Therefore, a problem associated with the prior art is that it has not been possible to develop an oral composition comprising a taxane in which the taxane has a high bioavailability with low variability. Clinical studies with oral paclitaxel [e.g. 3] and oral docetaxel [e.g. 9] have been executed by the inventors where the i.v. taxane formulations (also containing excipients such as Cremophor EL and ethanol, or polysorbate 80 and ethanol) were ingested orally. Nausea, vomiting and an unpleasant taste are frequently reported by the patients.

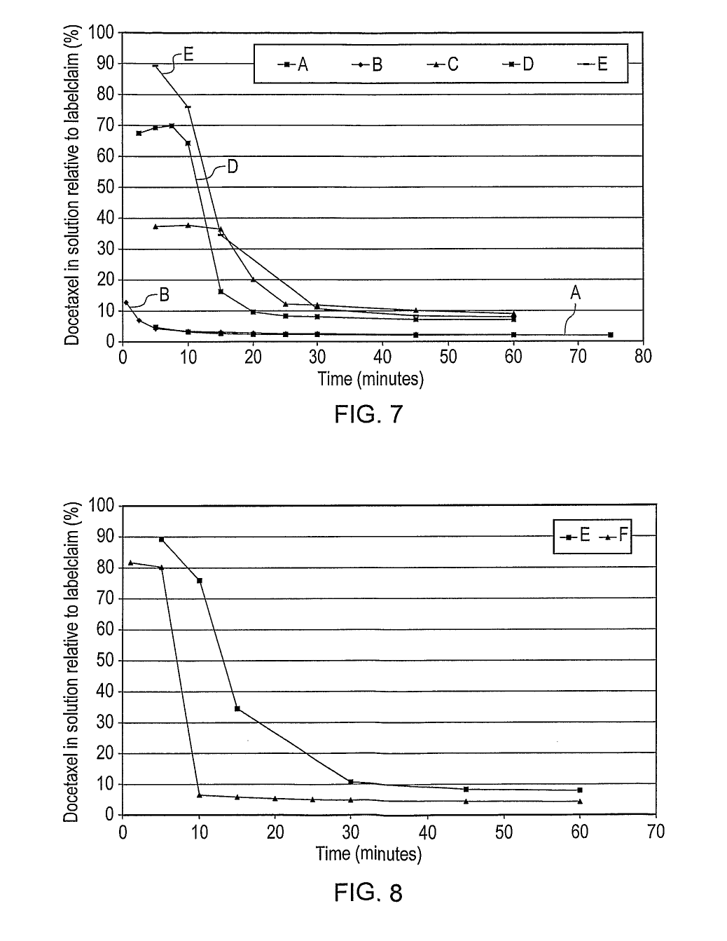

Chen et al. [13] conducted experiments to try to improve the solubility of the anticancer drug docetaxel in order to improve its bioavailability. Chen et al. tried using solid dispersions of docetaxel with various carriers, namely glyceryl monosterate, PVP-K30 or poloxamer 188. Chen et al. found that poloxamer 188 increased the solubility of docetaxel to about 3.3 μg/ml after 20 minutes (in a standard dissolution test) and to a maximum of about 5.5 μg/ml after about 120 minutes when a docetaxel to poloxamer ratio of 5:95 was used (see FIG. 7 in Chen paper). PVP-K30 only increased the solubility of docetaxel to about 0.8 μg/ml after 20 minutes and to a maximum of about 4.2 μg/ml after about 300 minutes (see FIG. 2). Glycerol monostearate hardly increased the solubility of docetaxel at all. Thus, the solubility and dissolution rate of docetaxel was not increased to a particularly high level. In order to achieve good oral bioavailability, a drug must have a relatively high solubility and dissolution rate so that there is a high enough amount of the drug in solution available for absorption within the first about 0.5 to 1.5 hours.

In a first aspect, the present invention provides a solid pharmaceutical composition for oral administration comprising a substantially amorphous taxane, a hydrophilic carrier and a surfactant, wherein the amorphous taxane is prepared by a solvent evaporation method.

The advantage provided by the composition of the invention is that the solubility of the taxane, the rate of dissolution of the taxane and/or the amount of time which the taxane remains in solution before starting to crystallise is increased to a surprising degree. These factors result in a significant increase in the bioavailability of the taxane. It is thought that this is due, at least in part, to the taxane being in a more amorphous state compared to the apparent amorphous taxane produced by other methods which are unlikely to be truely amorphous. Crystalline taxanes have very low solubilities.

The carrier helps to maintain the taxane in an amorphous state. Further, when the taxane is placed in aqueous media, the carrier helps to maintain the taxane in a supersaturated state in solution. This helps to stop the taxane from crystallising or increases the length of time before the taxane starts to crystallise in solution. Therefore, the solubility and dissolution rate of the taxane remain high. Further, the carrier gives good physical and chemical stability to the composition. It helps to prevent the degradation of the taxane and also helps to prevent the substantially amorphous taxane from changing to a more crystalline structure over time in the solid state. The good physical stability ensures the solubility of the taxane remains high.

The surfactant also helps to maintain the taxane in an amorphous state when placed in aqueous media and, surprisingly, substantially increases the solubility of the taxane compared to compositions comprising an amorphous taxane and a carrier.

The term “substantially amorphous” means that there should be little or no long range order of the position of the taxane molecules. The majority of the molecules should be randomly orientated. A completely amorphous structure has no long range order and contains no crystalline structure whatsoever; it is the opposite of a crystalline solid. However, it can be hard to obtain a completely amorphous structure for some solids. Therefore, many “amorphous” structures are not completely amorphous but still contain a certain amount of long range order or crystallinity. For example, a solid may be mainly amorphous but have pockets of crystalline structure or may contain very small crystals so that it is bordering on being truly amorphous. Therefore, the term “substantially amorphous” encompasses solids which have some amorphous structure but which also have some crystalline structure as well. The crystallinity of the substantially amorphous taxane should be less than 50%. Preferably, the crystallinity of the substantially amorphous taxane is less than 40%, even more preferably, less than 30%, more preferably still, less than 25%, even more preferably, less than 20%, more preferably still, less than 15%, even more preferably, less than 12.5%, more preferably still, less than 10%, even more preferably, less than 7.5%, more preferably still, less than 5% and most preferably, less than 2.5%. Since crystalline taxanes have low solubility, the lower the crystallinity of the substantially amorphous taxane, the better the solubility of the substantially amorphous taxane.

The substantially amorphous taxane can be prepared using any suitable solvent evaporation method. Suitable solvent evaporation methods are, for example, spray drying and vacuum drying as described in [18]. Preferably, the solvent evaporation method is spray drying. Surprisingly, it has been found that preparing the amorphous taxane using a solvent evaporation method, in particular spray drying, results in the composition having a particularly good solubility, dissolution rate and/or remains in solution for longer before starting to crystallise compared to compositions prepared using other methods. This is thought to be due to the solvent evaporation method producing a more amorphous taxane compared to other methods.

The composition for oral administration is in a solid form. The solid composition can be in any suitable form as long as the taxane is in a substantially amorphous state. For example, the composition can comprise a physical mixture of amorphous taxane, carrier and surfactant. In certain embodiments, the carrier and/or the surfactant are also in a substantially amorphous state. Preferably, the taxane and carrier are in the form of a solid dispersion. The term “solid dispersion” is well known to those skilled in the art and means that the taxane is partly molecularly dispersed in the carrier. More preferably, the taxane and carrier are in the form of a solid solution. The term “solid solution” is well known to those skilled in the art and means that the taxane is substantially completely molecularly dispersed in the carrier. It is thought that solid solutions are more amorphous in nature than solid dispersions. Methods of preparing solid dispersions and solid solutions are well known to those skilled in the art [11, 12]. Using these methods, both the taxane and carrier are in an amorphous state. When the taxane and carrier are in the form of a solid dispersion or solution, the solubility and dissolution rate of the taxane is greater than a physical mixture of amorphous taxane and carrier. It is thought that, when the taxane is in a solid dispersion or solution, the taxane is in a more amorphous state compared to apparently amorphous taxane on its own. It is thought that this results in the improved solubility and dissolution. The crystallinity of the solid dispersion or solution should be less than 50%. Preferably, the crystallinity of the solid dispersion or solution is less than 40%, even more preferably, less than 30%, more preferably still, less than 25%, even more preferably, less than 20%, more preferably still, less than 15%, even more preferably, less than 12.5%, more preferably still, less than 10%, even more preferably, less than 7.5%, more preferably still, less than 5% and most preferably, less than 2.5%.

When the taxane and carrier are in a solid dispersion, the surfactant can be in a physical mixture with the solid dispersion or solution. Preferably, however, the composition comprises a taxane, carrier and surfactant in the form of a solid dispersion or, more preferably, a solid solution. The advantage of having all three components in a solid dispersion or solution is that it enables the use of a lower amount of surfactant to achieve the same improvement in solubility and dissolution rate. Preferably, the taxane, carrier and surfactant are all in a substantially amorphous state.

Solid dispersions or solid solutions of taxane and carrier; or taxane, carrier and surfactant, can be produced using any suitable solvent evaporation method, as described above. Preferably, the solid dispersion or solid solution is prepared by spray drying. Surprisingly, it has been found that preparing the solid dispersion or solid solution using a solvent evaporation method, in particular spray drying, results in the composition having particularly good solubility characteristics so that the taxane has a good solubility, dissolution rate and/or remains in solution for longer before starting to crystallise compared to compositions prepared using other methods. This is thought to be due to the solvent evaporation method producing a composition in which all the components are in a more amorphous state compared to other methods. In other methods, it has been found that one or more of the components may still have some crystalline nature which is thought to result in the composition having reduced solubility characteristics and/or physical stability in solution.

In one embodiment, the composition can be contained in a capsule for oral administration. The capsule can be filled in a number of different ways. For example, the amorphous taxane may be prepared by spray drying, powdered, combined with the carrier and surfactant, and then dispensed into the capsule.

In an alternative embodiment, the composition can be compressed into tablets. For example, the amorphous taxane may be prepared by spray drying, powdered, mixed with the carrier and surfactant (and optionally other excipients), and then an appropriate amount compressed into a tablet.

Taxanes are diterpene compounds which originate from plants of the genus Taxus (yews). However, some taxanes have now been produced synthetically or semi synthetically. Taxanes inhibit cell growth by stopping cell division and are used in treatment of cancer. They stop cell division by disrupting microtubule formation. They may also act as angiogenesis inhibitors. The term “taxane”, as used herein, includes all diterpene taxanes, whether produced naturally or artificially, functional derivatives and pharmaceutically acceptable salts or esters which bind to tubulin.

Derivatives of taxanes containing groups to modify physiochemical properties are also included within the present invention. Thus, polyalkylene glycol (such as polyethylene glycol) or saccharide conjugates of taxanes, with improved or modified solubility characteristics, are included.

The taxane of the composition can be any suitable taxane as defined above. Preferred taxanes are docetaxel, paclitaxel, BMS-275183, functional derivatives thereof and pharmaceutically acceptable salts or esters thereof. BMS-275183 is a C-3′-t-butyl-3′-N-t-butyloxycarbonyl analogue of paclitaxel [10]. More preferably, the taxane is selected from docetaxel, paclitaxel, functional derivatives thereof and pharmaceutically acceptable salts or esters thereof.

The hydrophilic carrier of the composition is an organic compound capable of at least partial dissolution in aqueous media at pH 7.4 and/or capable of swelling or gelation in such aqueous media. The carrier can be any suitable hydrophilic carrier which ensures that the taxane remains in an amorphous state in the composition and increases the solubility and dissolution rate of the taxane. Preferably, the carrier is polymeric. Preferably, the carrier is selected from: polyvinylpyrrolidone (PVP); polyethylene glycol (PEG); polyvinylalcohol (PVA); crospovidone (PVP-CL); polyvinylpyrrolidone-polyvinylacetate copolymer (PVP-VA); cellulose derivatives such as methylcellulose, hydroxypropylcellulose, carboxymethylethylcellulose, hydroxypropylmethylcellulose (HPMC), cellulose acetate phthalate and hydroxypropylmethylcellulose phthalate; polyacrylates; polymethacrylates; sugars, polyols and their polymers such as mannitol, sucrose, sorbitol, dextrose and chitosan; and cyclodextrins. More preferably, the carrier is selected from PVP, PEG and PVP-VA, more preferably still, the carrier is selected from PVP and PVP-VA. In one embodiment, the carrier is PVP. In an alternative embodiment, the carrier is PVP-VA.

If the carrier is PVP, it can be any suitable PVP [16] to act as a carrier and to help keep the taxane in an amorphous state. For example, the PVP may be selected from PVP-K12, PVP-K15, PVP-K17, PVP-K25, PVP-K30, PVP-K60, PVP-K90 and PVP-K120. Preferably, the PVP is selected from PVP-K30, PVP-K60 and PVP-K90. Most preferably, the PVP is PVP-K30.

If the carrier is PEG, it can be any suitable PEG [16] to act as a carrier and to help keep the taxane in an amorphous state. For example, the PEG may be selected from PEG1500, PEG6000 and PEG20000. Preferably, the PEG is selected from PEG1500 and PEG6000, and most preferably, the PEG is PEG1500.

If the carrier is PVP-VA, it can be any suitable PVP-VA [16] to act as a carrier and to help keep the taxane in an amorphous state. For example, the PVP-VA may be PVP-VA 64.

The composition can contain any suitable amount of the carrier relative to the amorphous taxane so that the carrier maintains the amorphous taxane in its amorphous state. Preferably, the taxane to carrier weight ratio is between about 0.01:99.99 w/w and about 75:25 w/w. More preferably, the taxane to carrier weight ratio is between about 0.01:99.99 w/w and about 50:50 w/w, even more preferably, between about 0.01:99.99 w/w and about 40:60 w/w, more preferably still, between about 0.01:99.99 w/w and about 30:70 w/w, even more preferably, between about 0.1:99.9 w/w and about 20:80 w/w, more preferably still, between about 1:99 w/w and about 20:80 w/w, even more preferably, between about 2.5:97.5 w/w and about 20:80 w/w, more preferably still, between about 2.5:97.5 w/w and about 15:85 w/w, even more preferably, between about 5:95 w/w and about 15:85 w/w and most preferably, about 10:90 w/w.

The surfactant can be any suitable pharmaceutically acceptable surfactant and such surfactants are well known to those skilled in the art. For example, the surfactant can be an anionic, cationic or non-ionic surfactant. Preferably, the surfactant is a cationic or anionic surfactant. More preferably, the surfactant is an anionic surfactant.

In one embodiment, the surfactant preferably has an HLB (hydrophilic lipophilic balance) value of greater than about 2. More preferably, the HLB value is grater than about 4, more preferably still, the HLB value is greater than about 10, even more preferably, the HLB value is greater than about 14, more preferably still the HLB value is greater than about 20, even more preferably, the HLB value is greater than about 25, more preferably still the HLB value is greater than about 30, and most preferably, the HLB value is greater than about 35. Preferably, the HLB value should be less than about 45.

Preferably, the surfactant is selected from triethanolamine, sunflower oil, stearic acid, monobasic sodium phosphate, sodium citrate dihydrate, propylene glycol alginate, oleic acid, monoethanolamine, mineral oil and lanolin alcohols, methylcellulose, medium-chain triglycerides, lecithin, hydrous lanolin, lanolin, hydroxypropyl cellulose, glyceryl monostearate, ethylene glycol pamitostearate, diethanolamine, lanolin alcohols, cholesterol, cetyl alcohol, cetostearyl alcohol, castor oil, sodium dodecyl sulphate (SDS), sorbitan esters (sorbitan fatty acid esters), polyoxyethylene stearates, polyoxyethylene sorbitan fatty acid esters, polyoxyethylene castor oil derivatives, polyxoyethylene alkyl ethers, poloxamer, glyceryl monooleate, docusate sodium, cetrimide, benzyl benzoate, benzalkonium chloride, benzethonium chloride, hypromellose, non-ionic emulsifying wax, anionic emulsifying wax and triethyl citrate (these compounds are indicated as being emulsifiers and surfactants in the Handbook of Pharmaceutical Excipients (4^{th} Edition, editors: R C Rowe, P J Sheskey, P J Weller)). More preferably, the surfactant is selected from sodium dodecyl sulphate (SDS), sorbitan esters (sorbitan fatty acid esters), polyoxyethylene stearates, polyoxyethylene sorbitan fatty acid esters, polyoxyethylene castor oil derivatives, polyxoyethylene alkyl ethers, poloxamer, glyceryl monooleate, docusate sodium, cetrimide, benzyl bezoate, benzalkonium chloride, benzethonium chloride, hypromellose, non-ionic emulsifying wax, anionic emulsifying wax and triethyl citrate (these compounds are indicated as being surfactants in the Handbook of Pharmaceutical Excipients (4^{th} Edition, editors: R C Rowe, P J Sheskey, P J Weller)). More preferably, the surfactant is selected from sodium dodecyl sulphate (SDS), sorbitan esters (sorbitan fatty acid esters), and polyoxyethylene sorbitan fatty acid esters. In one embodiment, the surfactant can be cetylpyridinium chloride (CPC). In another embodiment, the surfactant is selected from SDS, CPC, polyoxyethylene (20) sorbitan monooleate (polysorbate 80) and polysorbitan monooleate. Preferably, the surfactant is selected from SDS, CPC and polysorbate 80. More preferably, the surfactant is selected from SDS and CPC. Most preferably, the surfactant is SDS.

Any suitable amount of surfactant can be used in the composition in order to improve the solubility and dissolution rate of the taxane. Preferably, the weight ratio of surfactant, to taxane and carrier combined, is between about 1:99 w/w and about 50:50 w/w, more preferably, between about 1:99 w/w and about 44:56 w/w, even more preferably, between about 1:99 w/w and about 33:67 w/w, more preferably still, between about 2:98 w/w and about 33:67 w/w, even more preferably, between about 2:98 w/w and about 17:83 w/w, more preferably still, between about 5:95 w/w and about 17:83 w/w and most preferably, about 9:91 w/w.

Alternatively, the weight ratio of surfactant to taxane is preferably between about 1:100 w/w and about 60:1 w/w, more preferably, between about 1:50 w/w and about 40:1 w/w, even more preferably, between about 1:20 w/w and about 20:1 w/w, more preferably still, between about 1:10 w/w and about 10:1 w/w, even more preferably, between about 1:5 w/w and about 5:1 w/w, more preferably still, between about 1:3 w/w and about 3:1 w/w, even more preferably, between about 1:2 w/w and about 2:1 w/w and most preferably, about 1:1 w/w.

In one embodiment, the composition comprises an enteric coating. Any suitable enteric coating can be used, for example, cellulose acetate phthalate, polyvinyl acetate phthalate and suitable acrylic derivates, e.g. polymethacrylates. An enteric coating prevents the release of the taxane in the stomach and thereby prevents acid-mediated degradation of the taxane. Furthermore, it enables targeted delivery of the taxane to the intestines where the taxane is absorbed, thus ensuring that the limited time during which the taxane is present in solution (before crystallisation takes place) is only spent at sites where absorption is possible.

In one embodiment, the composition may further comprise one or more additional pharmaceutically active ingredients. Preferably, one or more of the additional pharmaceutically active ingredients is a CYP3A4 inhibitor. Suitable CYP3A4 inhibitors are grapefruit juice or St. John's wort (or components of either), ritonavir, lopinavir or imidazole compounds, such as ketoconazole. Preferably, the CYP3A4 inhibitor is ritonavir.

Where the composition comprises one or more additional pharmaceutically active ingredients, the pharmaceutically active ingredient(s) can be included into the composition as a physical mixture. Alternatively, the pharmaceutically active ingredient(s) can be in an amorphous form. The pharmaceutically active ingredient(s) can be in an amorphous form in a physical mixture with the other amorphous and/or non-amorphous components. Alternatively, it can be in a solid dispersion, or preferably a solid solution, with the taxane; with the taxane and carrier; or with the taxane, carrier and surfactant. When the additional pharmaceutically active ingredient(s) is in an amorphous state, or is in a solid dispersion or solid solution, it should be prepared using a solvent evaporation method, for example, spray drying.

When the composition is in a tablet form and comprises one or more additional pharmaceutically active ingredients, the one or more additional pharmaceutically active ingredients are preferably in the same tablet as the amorphous taxane, i.e. in a single tablet with the other components.

The pharmaceutical composition may comprise additional pharmaceutically acceptable excipients, adjuvants and vehicles which are well known to those skilled in the art. Pharmaceutically acceptable excipients, adjuvants and vehicles that may be used in the pharmaceutical compositions of this invention include, but are not limited to, ion exchangers, alumina, aluminium stearate, serum proteins, such as human serum albumin, buffer substances such as phosphates, glycerine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate and wool fat.

The pharmaceutical compositions can be orally administered in any orally acceptable dosage form including, but not limited to, capsules, tablets, a powder or coated granules. Tablets may be formulated to be immediate release, extended release, repeated release or sustained release. They may also, or alternatively, be effervescent, dual-layer and/or coated tablets. Capsules may be formulated to be immediate release, extended release, repeated release or sustained release. Lubricating agents, such as magnesium stearate, are also typically added. For oral administration in a capsule form, useful diluents include lactose and dried corn starch. For tablets and capsules, other pharmaceutical excipients that can be added are binders, fillers, filler/binders, adsorbents, moistening agents, disintegrants, lubricants, glidants, and the like. Tablets and capsules may be coated to alter the appearance or properties of the tablets and capsules, for example, to alter the taste or to colour coat the tablet or capsule.

Other pharmaceutically acceptable additives which may be added to the composition are well known to those skilled in the art.

The present invention also provides the above composition for use in therapy.

Further, the present invention provides the above composition for use in the treatment of neoplastic disease.

The neoplastic disease treated by the present invention is preferably a solid tumour. The solid tumour is preferably selected from breast, lung, gastric, colorectal, head & neck, oesophageal, liver, renal, pancreatic, bladder, prostate, testicular, cervical, endometrial, ovarian cancer and non-Hodgkin's lymphoma (NHL). The solid tumour is more preferably selected from breast, gastric, ovarian, prostate, head & neck and non-small cell lung cancer.

Further neoplastic diseases that may be treated by the present invention are multiple myeloma, chronic myelomonocytic leukaemia (CMML), acute myeloid leukaemia (AML) and Kapsoi's sarcoma. Furthermore, the disease range includes myelodysplastic syndromes (MDS).

The present invention also provides a method of treatment of a neoplastic disease, the method comprising the administration, to a subject in need of such treatment, of an effective amount of the above composition.

Preferably, the method is used to treat a human subject.

The present invention also provides a method of preparing the above composition comprising the steps of:
preparing an amorphous taxane using a solvent evaporation method; and
combining the amorphous taxane with a hydrophilic carrier and a surfactant to produce the composition.

The amorphous taxane can be produced by any suitable solvent evaporation method, for example, as described above. Preferably, the amorphous taxane is produced by spray drying.

The preparation of the amorphous taxane, and the combining thereof with the carrier and/or surfactant may be carried out in a single step, e.g. where the taxane and the carrier and/or the surfactant are subjected to amorphosing treatment together (for example to form a solid dispersion or solution).

Preferably, the method comprises the steps of preparing a solid dispersion comprising the taxane and the hydrophilic carrier, and combining the solid dispersion with the surfactant.

More preferably, the method comprises the step of preparing a solid dispersion comprising the taxane, the hydrophilic carrier and the surfactant.

In another aspect, the present invention provides a pharmaceutical composition for oral administration comprising a substantially amorphous taxane and a hydrophilic carrier, wherein the substantially amorphous taxane is prepared by spray drying.

The present invention also provides a method of preparing a solid pharmaceutical composition for oral administration comprising a substantially amorphous taxane and a hydrophilic carrier, the method comprising the steps of:
preparing an amorphous taxane by spray drying; and
combining the amorphous taxane with a hydrophilic carrier to produce the composition.

The advantage provided by this composition is that the solubility of the taxane, the rate of dissolution of the taxane and/or the amount of time which the taxane remains in solution before starting to crystallise is increased to a surprising degree. It is thought that this is because the solvent evaporation method produces an even more amorphous taxane compared to other methods of producing amorphous taxanes in which the taxane is unlikely to be truely amorphous. It is thought that the more amorphous nature of the taxane provides the increased solubility characteristics.

Additional optional features of the composition are the same as for the composition comprising an amorphous taxane, a carrier and a surfactant. For example, the composition comprising a substantially amorphous taxane and a carrier, wherein the substantially amorphous taxane is prepared by spray drying, preferably further comprises a surfactant. The preferred embodiments of the taxane, the carrier, the crystallinity of the taxane, the ratio of taxane to carrier, the state of the taxane and carrier, etc. are as defined above.

In another aspect, the present invention also provides a pharmaceutical composition comprising a taxane, a hydrophilic carrier and a surfactant, in solution. The description above relating to the identity, properties, etc. of the taxane, carrier and surfactant are equally applicable to this aspect of the invention. In such a composition, all three components of the composition are in solution.

The pharmaceutical composition may be in the form of a drinking solution for administration to a subject. Alternatively, the pharmaceutical composition can be placed in capsules, for example, gelatin capsules to form liquid filled capsules containing the solution.

The composition can be obtained by dissolving the solid composition described above in a suitable solvent. Therefore, in one embodiment, the composition is obtainable by dissolving the solid composition described above in a suitable solvent such as triacetin. In one embodiment, the solvent is an aqueous solvent.

In another aspect, the invention provides a solid pharmaceutical composition for oral administration comprising a substantially amorphous taxane and one or more pharmaceutically acceptable excipients, wherein the substantially amorphous taxane is prepared by spray drying.

The characteristics and preferred features of this composition are as described above for the other compositions of the invention.

The present invention will now be described by way of example only with reference to the accompanying figures in which:

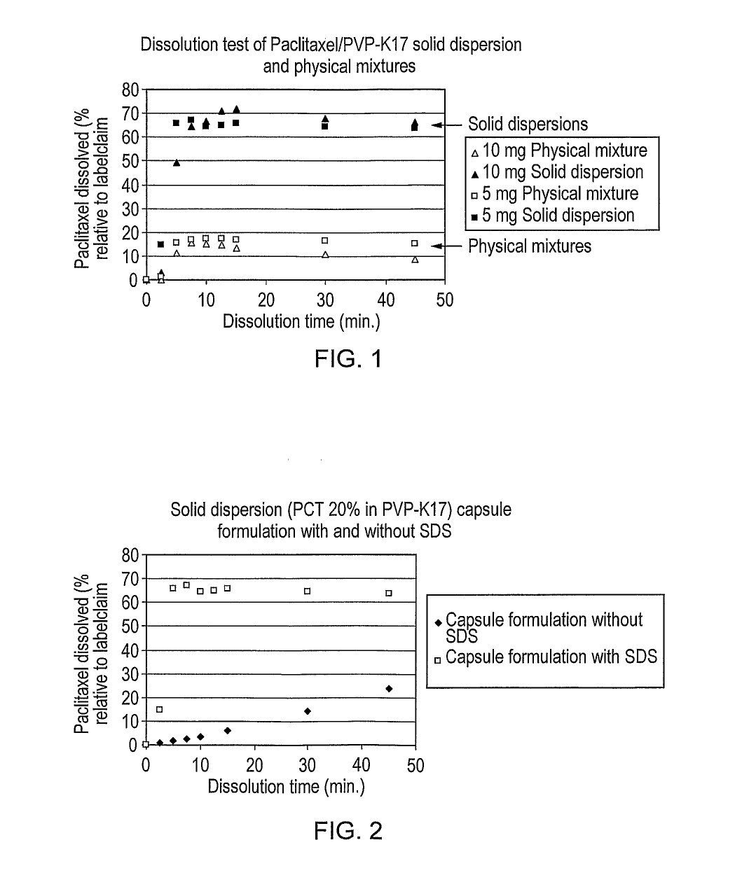

FIG. 1 shows the results of a dissolution test of paclitaxel solid dispersions versus paclitaxel physical mixtures (conditions: 900 mL WfI, 37° C., 75 rpm);

FIG. 2 shows the results of a dissolution test of paclitaxel (PCT) solid dispersion capsules with and without sodium dodecyl sulphate (conditions: 900 mL WfI, 37° C., 75 rpm);

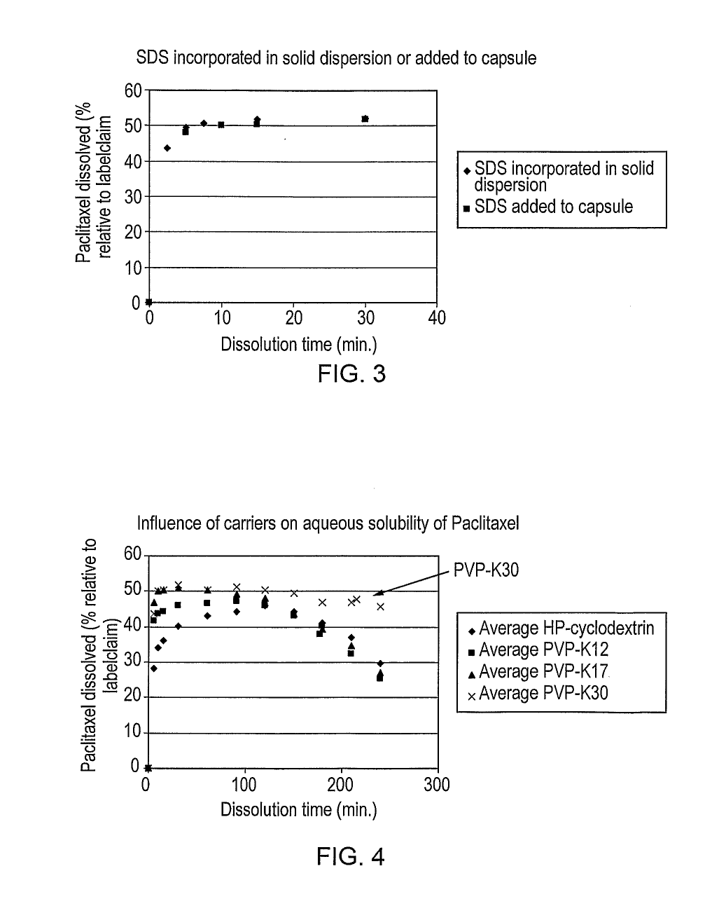

FIG. 3 shows the results of a dissolution test of paclitaxel solid dispersions with sodium dodecyl sulphate incorporated in the solid dispersion or added to the capsule (conditions: 500 mL WfI, 37° C., 75 rpm (100 rpm for SDS added to the capsules));

FIG. 4 shows the results of a dissolution test of paclitaxel solid dispersions with various carriers (conditions: 500 mL WfI, 37° C., 100 rpm);

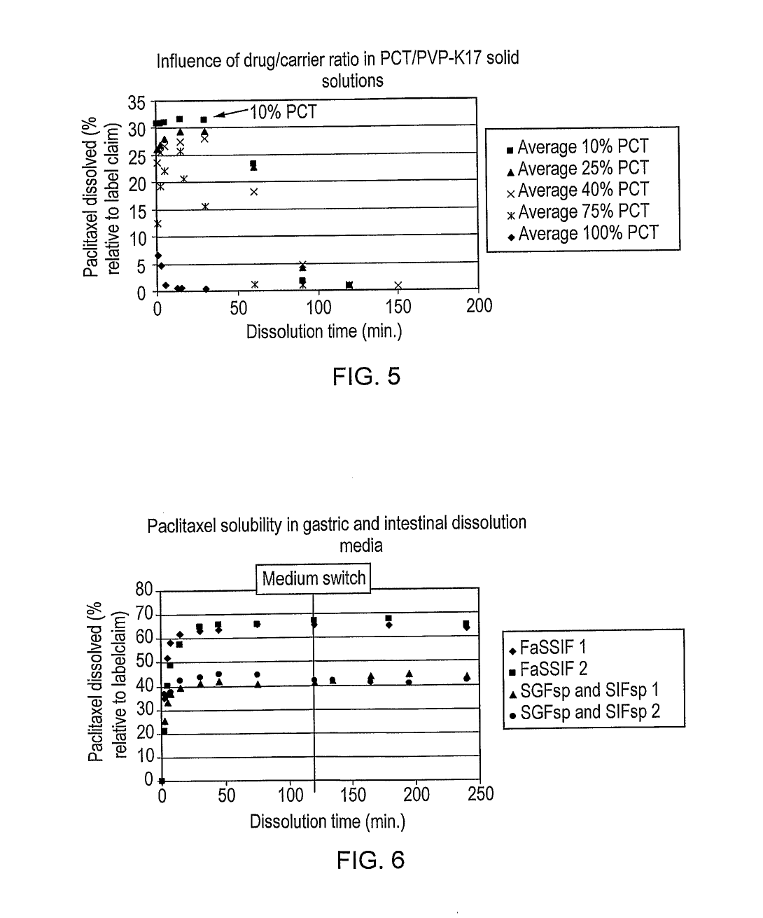

FIG. 5 shows the results of a solubility test of paclitaxel/PVP-K17 solid dispersions with various drug-carrier ratios (conditions: 25 mL WfI, 37° C., 7200 rpm);

FIG. 6 shows the results of a dissolution test of paclitaxel solid dispersions in various media (conditions: 500 mL FaSSIF (light grey), 37° C., 75 rpm; or 500 mL SGFₛₚ and 629 mL SIFₛₚ, 37° C., 75 rpm (dark grey));

FIG. 7 shows the docetaxel solubility of five different formulations (see table 13). A: anhydrous docetaxel; B: amorphous docetaxel; C: physical mixture of anhydrous docetaxel, PVP-K30 and SDS; D: physical mixture of amorphous docetaxel, PVP-K30 and SDS; E: solid dispersion of amorphous docetaxel, PVP-K30 and SDS (dissolution conditions: ±6 mg docetaxel, 25 mL WfI, 37° C., 720 rpm);

FIG. 8 shows docetaxel solubility of solid dispersions with different carriers (see table 13). E: Solid dispersion of amorphous docetaxel, PVP-K30 and SDS; F: Solid dispersion of amorphous docetaxel, HPβ-CD and SDS. (Dissolution conditions: ±6 mg Docetaxel, 25 mL WfI, 37° C., 720 rpm);

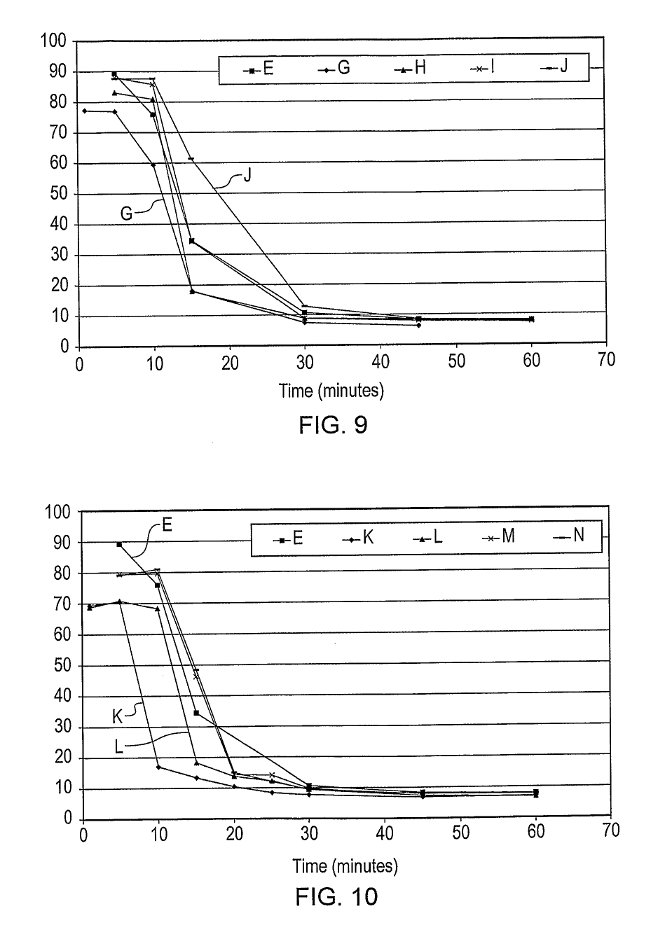

FIG. 9 shows docetaxel solubility of solid dispersions with PVP of various chain lengths (see table 13). E: solid dispersion of amorphous docetaxel, PVP-K30 and SDS; G: solid dispersion of amorphous docetaxel, PVP-K12 and SDS; H: solid dispersion of amorphous docetaxel, PVP-K17 and SDS; I: solid dispersion of amorphous docetaxel, PVP-K25 and SDS; J: solid dispersion of amorphous docetaxel, PVP-K90 and SDS. (Dissolution conditions: ±6 mg Docetaxel, 25 mL WfI, 37° C., 720 rpm);

FIG. 10 shows docetaxel solubility of solid dispersions with various drug loads (see table 13). E: 1/11 docetaxel; K: 5/7 docetaxel; L: 1/3 docetaxel; M: 1/6 docetaxel; N: 1/21 docetaxel. (Dissolution conditions: ±6 mg Docetaxel, 25 mL WfI, 37° C., 720 rpm);

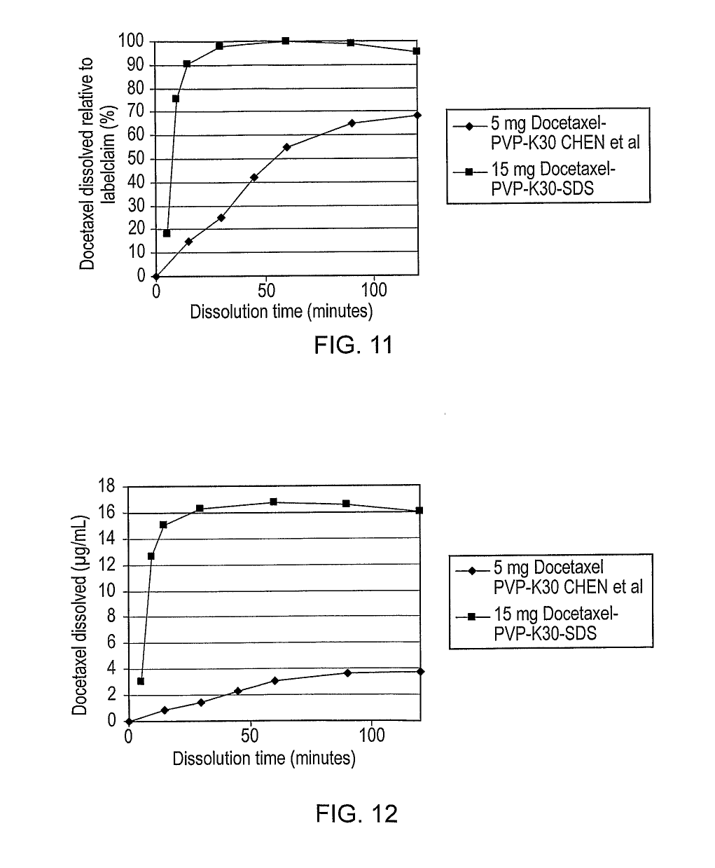

FIG. 11 shows the dissolution results in terms of the relative amount of docetaxel dissolved of a solid dispersion of docetaxel, PVP-K30 and SDS, compared to literature data of a solid dispersion of docetaxel and PVP-K30 (Chen et al., [13]);

FIG. 12 shows the dissolution results in terms of the absolute amount of docetaxel dissolved of a solid dispersion of docetaxel, PVP-K30 and SDS, compared to literature data of a solid dispersion of docetaxel and PVP-K30 (Chen et al., [13]);

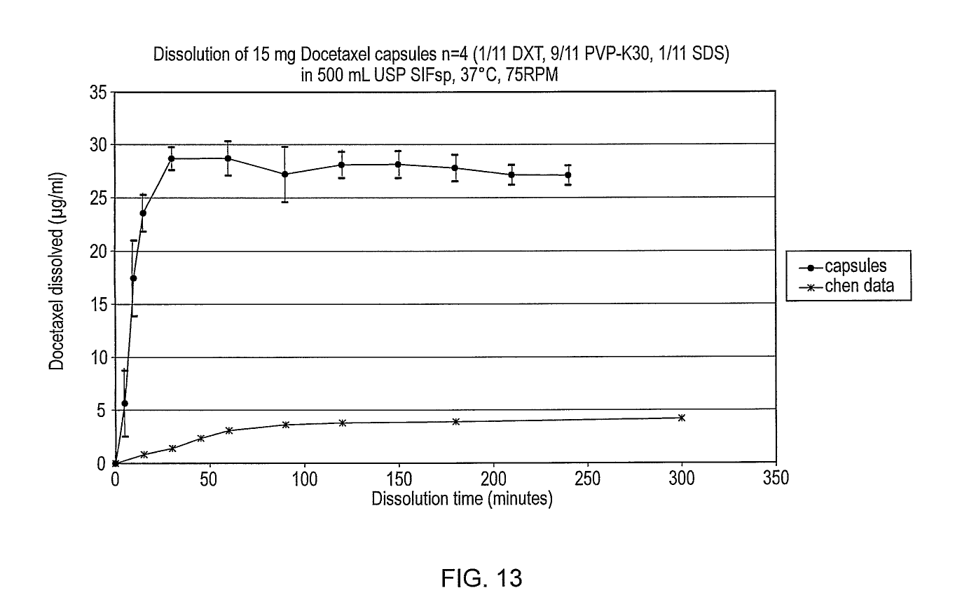

FIG. 13 shows the results of a dissolution test of docetaxel capsules (15 mg docetaxel (DXT) per capsule with PVP-K30+SDS) compared with literature data (Chen et al. [13].

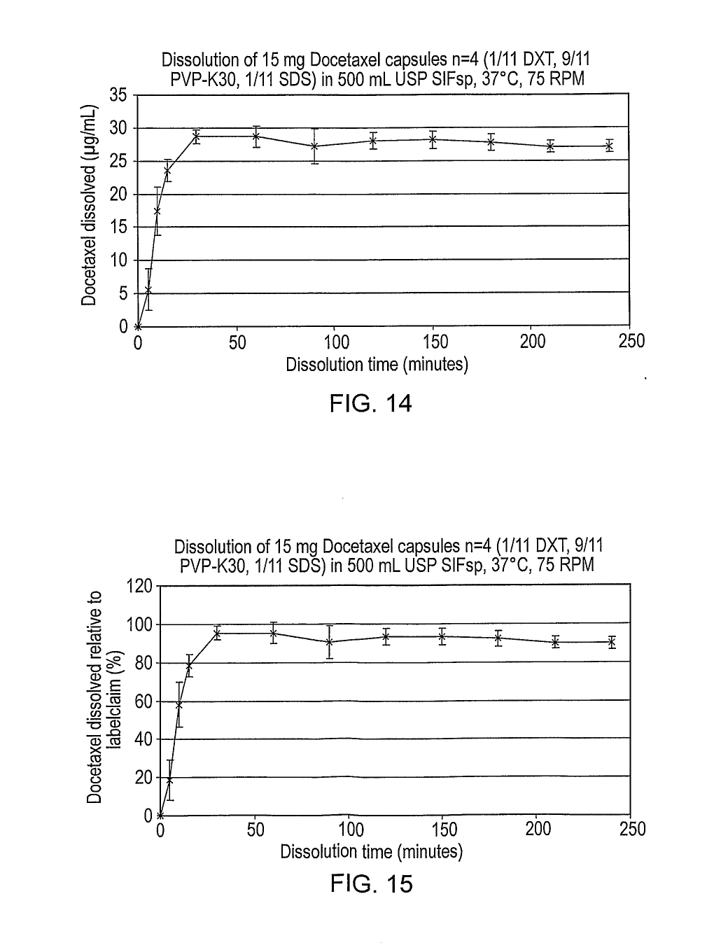

FIG. 14 shows the dissolution results in terms of the absolute amount of docetaxel dissolved of a solid dispersion of docetaxel, PVP-K30 and SDS. The dissolution test was carried out in Simulated Intestinal Fluid sine Pancreatin (SIFsp);

FIG. 15 shows the dissolution results in terms of the relative amount of docetaxel dissolved of a solid dispersion of docetaxel, PVP-K30 and SDS. The dissolution test was carried out in Simulated Intestinal Fluid sine Pancreatin (SIFsp);

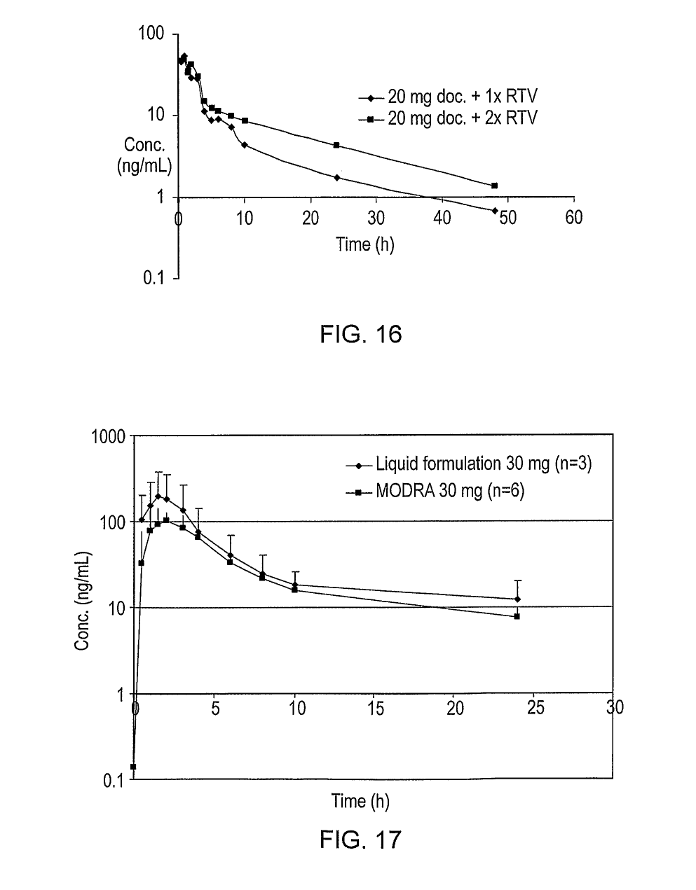

FIG. 16 shows the docetaxel pharmacokinetic curves of a patient who received docetaxel and ritonavir simultaneously in a first cycle. In the second cycle, the patient received docetaxel and ritonavir simultaneously at t=0 and then an additional booster dose of ritonavir at t=4 h;

FIG. 17 shows the pharmacokinetic curves of four patients who received a liquid formulation of docetaxel and/or a solid dispersion comprising docetaxel (referred to as MODRA);

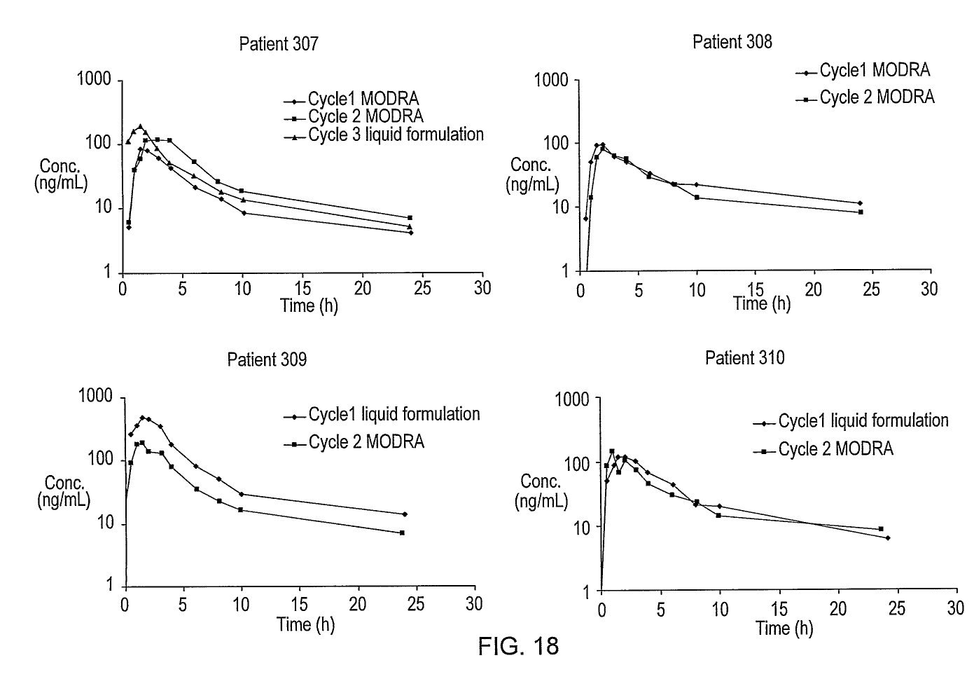

FIG. 18 shows the pharmacokinetic curves of patients receiving the liquid oral formulation of docetaxel compared to the patients receiving the solid oral formulation of docetaxel (MODRA); and

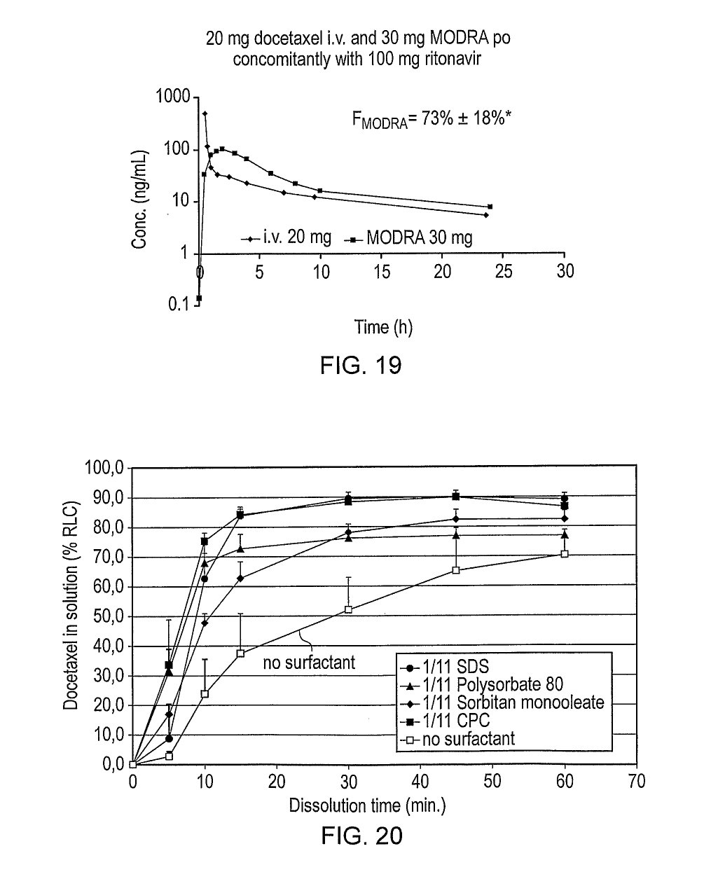

FIG. 19 shows pharmacokinetic curves after i.v. and oral administration of docetaxel. Both i.v. and oral docetaxel administration was combined with administration of ritonavir. N.B. The calculated bioavailability is corrected for the administered dose.

FIG. 20 shows the average dissolution curves of five docetaxel formulations with different types of surfactants (n=3).

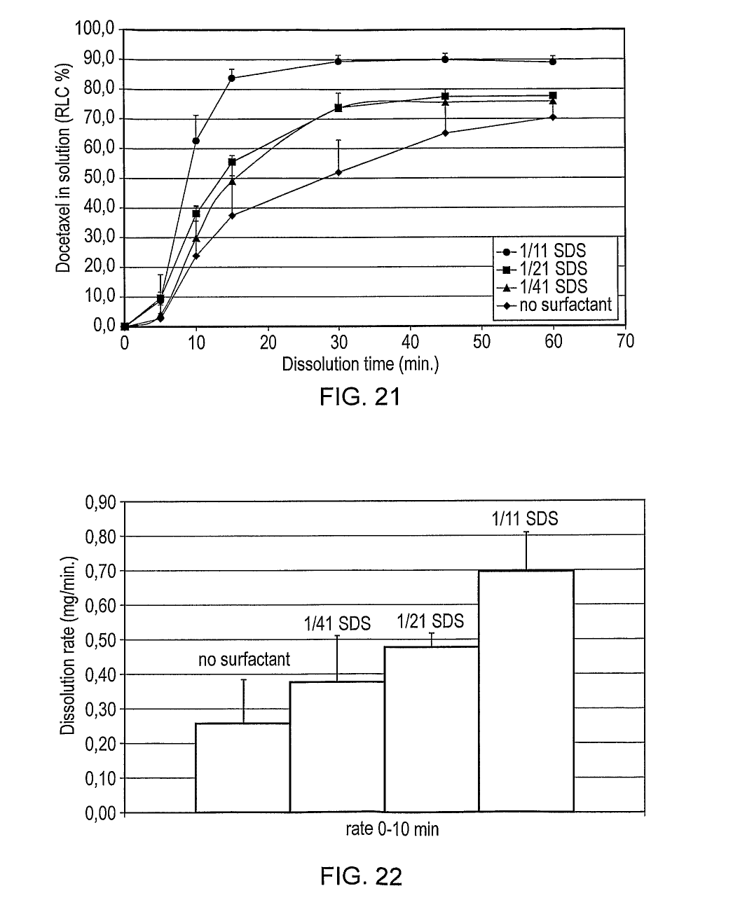

FIG. 21 shows the average dissolution curves of docetaxel formulations with various amounts of SDS surfactant (n=3).

FIG. 22 shows the average dissolution rates between 0 and 10 minutes of four docetaxel formulations with various amounts of surfactant (n=3).

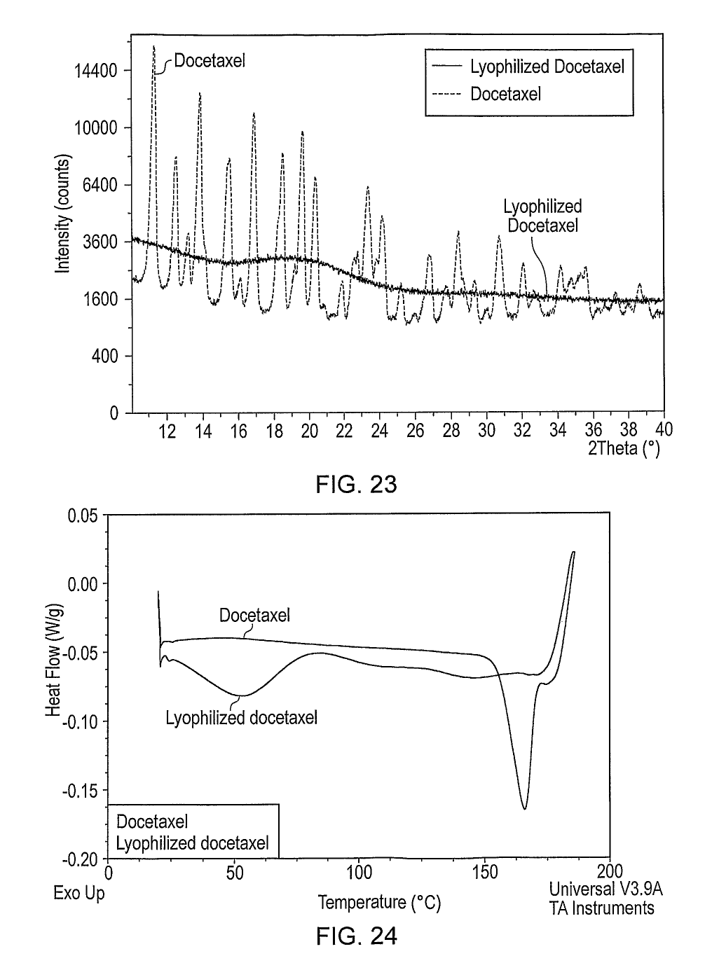

FIG. 23 shows the X-ray powder diffraction patterns of lyophilized and crystalline docetaxel.

FIG. 24 shows the DSC thermograms of lyophilized and crystalline docetaxel.

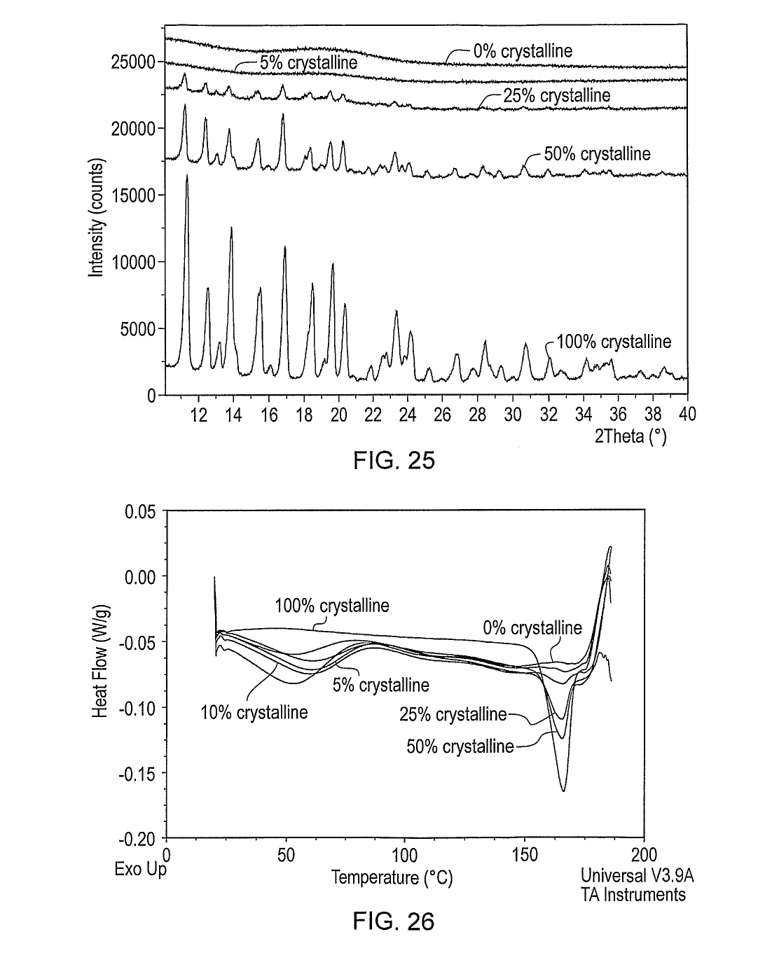

FIG. 25 shows the X-ray powder diffraction spectra of physical mixtures of crystalline and amorphous docetaxel.

FIG. 26 shows the DSC thermograms of physical mixtures of crystalline and amorphous docetaxel.

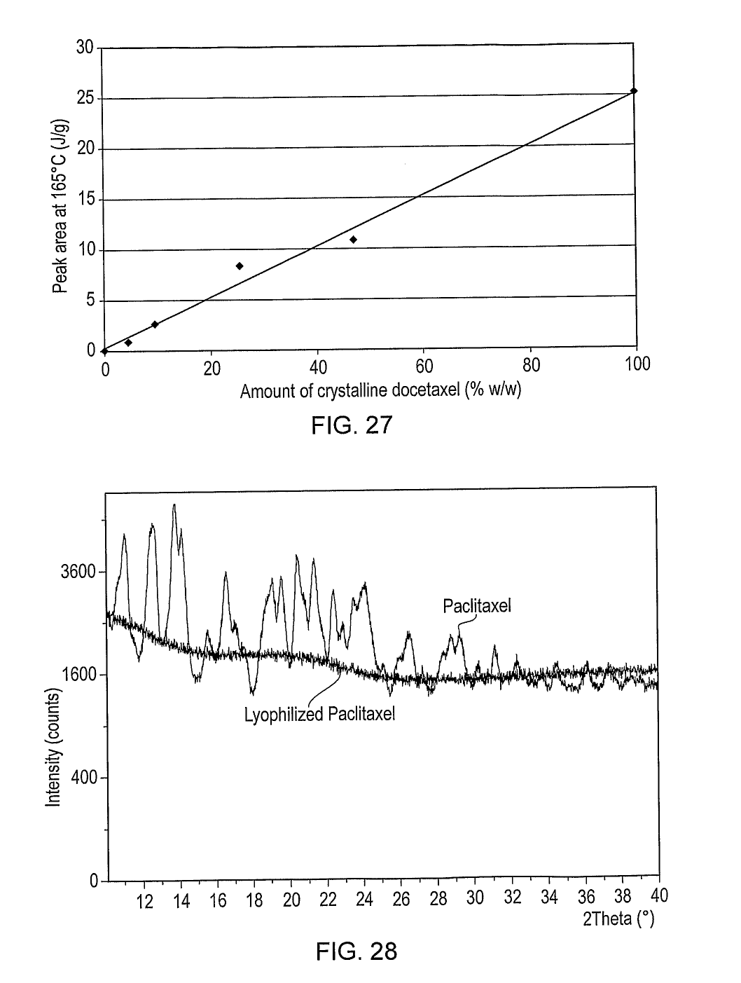

FIG. 27 shows the peak area at 165° C. in the total heat flow thermogram vs. the crystalline docetaxel content. The black line is the linear regression line with an R² of 0.990.

FIG. 28 shows the X-ray powder diffraction patterns of lyophilized and crystalline paclitaxel.

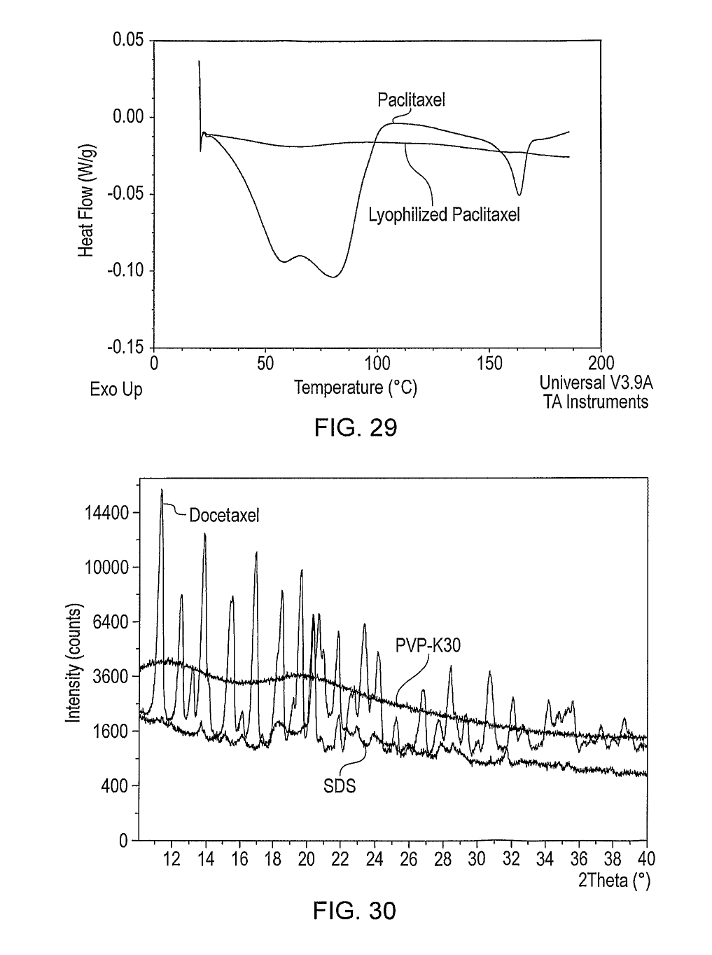

FIG. 29 shows a DSC thermogram of lyophilized and crystalline paclitaxel.

FIG. 30 shows X-ray powder diffraction patterns of docetaxel, PVP-K30 and SDS.

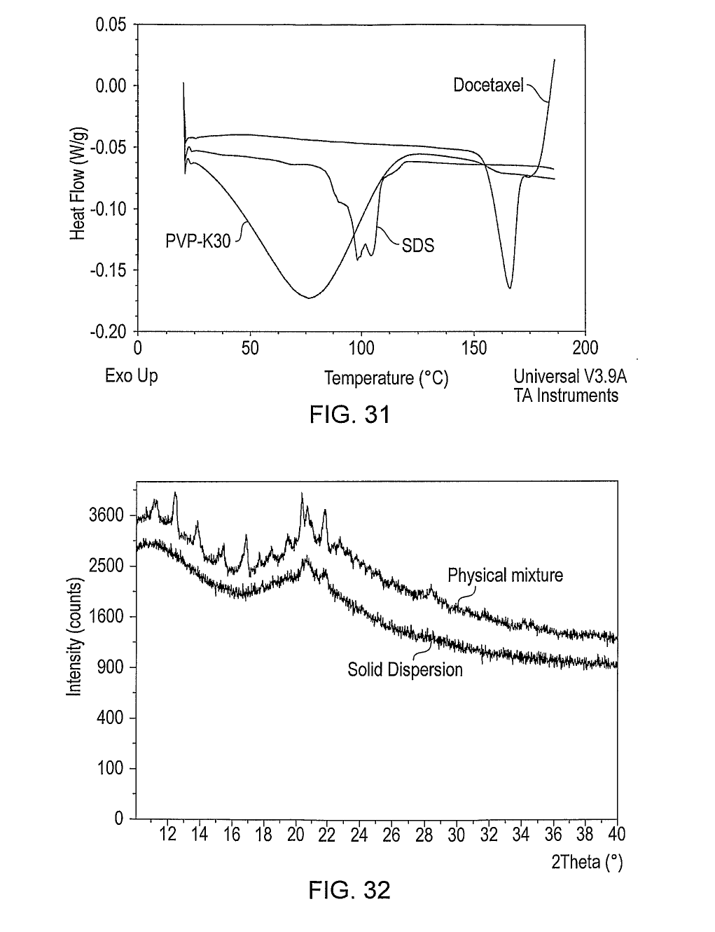

FIG. 31 shows DSC thermograms of PVP-K30, SDS and docetaxel.

FIG. 32 shows X-ray powder diffraction spectra of a physical mixture and solid dispersion with 1/11 docetaxel, 9/11 PVP-K30 and 1/11 SDS.

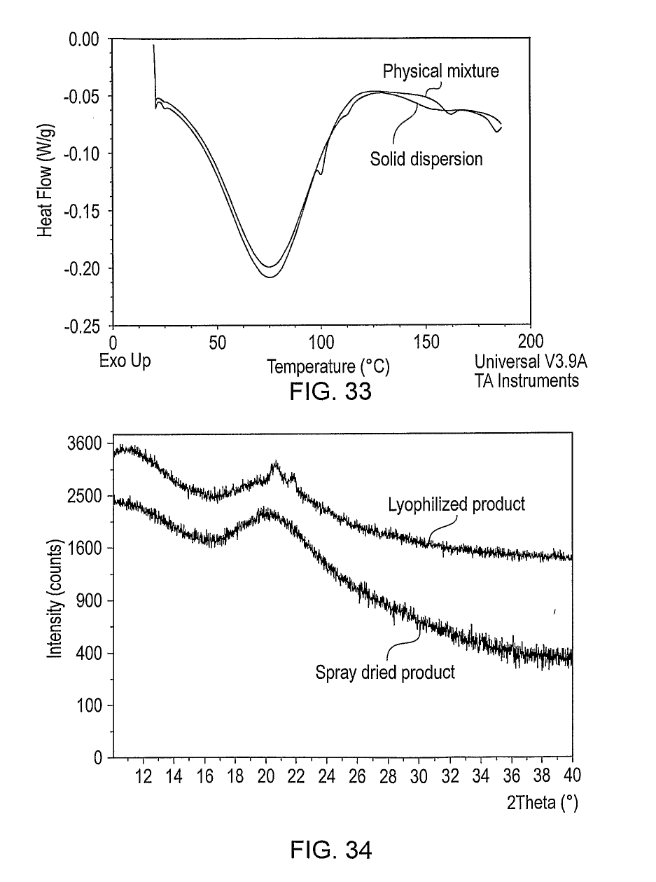

FIG. 33 shows DSC thermograms of a physical mixture and solid dispersion with 1/11 docetaxel, 9/11 PVP-K30 and 1/11 SDS.

FIG. 34 shows X-ray powder diffraction spectra of solid dispersions produced by lyophilization and spray drying.

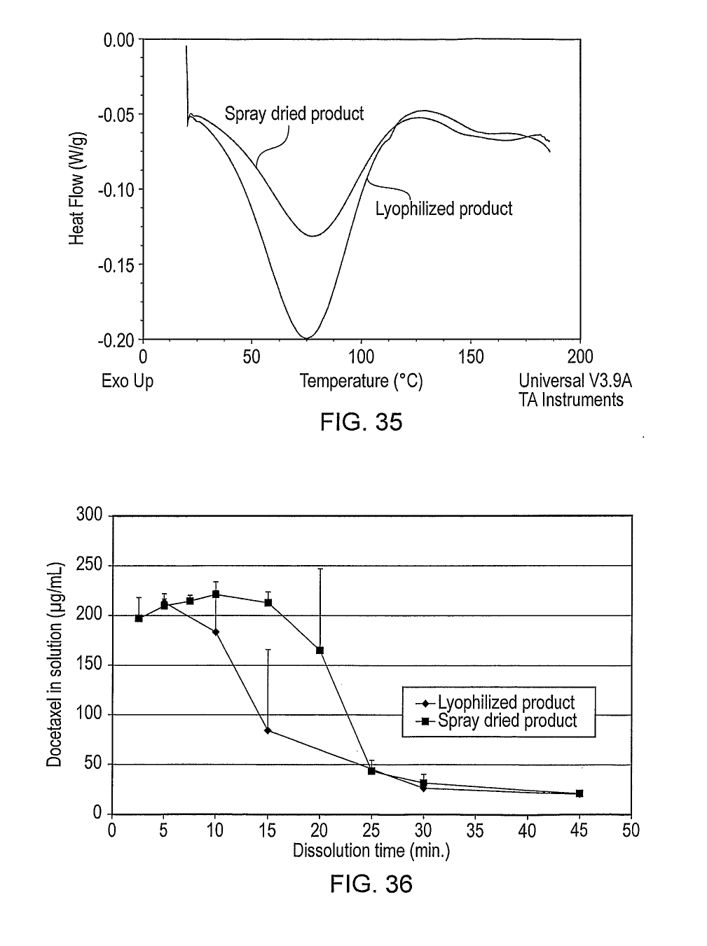

FIG. 35 shows DSC thermograms of solid dispersions produced by lyophilization and spray drying.

FIG. 36 shows the average dissolution screening curves of solid dispersions produced by lyophilization and spray drying (n=4).

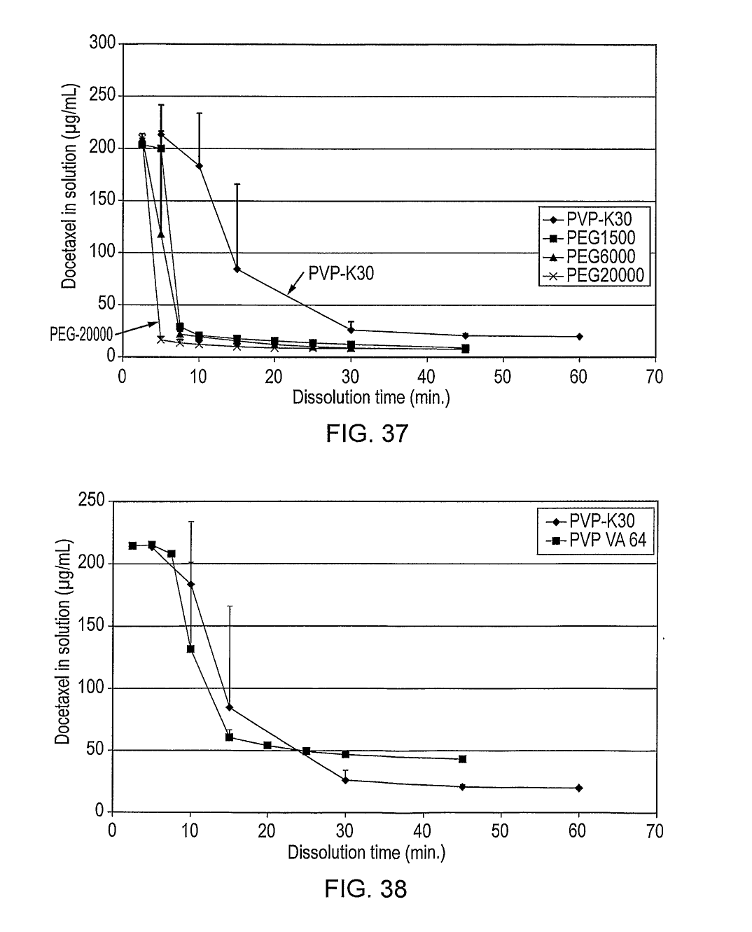

FIG. 37 shows the average dissolution screening curves of solid dispersions of docetaxel with PVP-K30 (n=4), PEG 1500 (n=2), PEG6000 (n=2) and PEG20000 (n=2).

FIG. 38 shows the average dissolution screening curves of solid dispersions of docetaxel with PVP-K30 (n=4) and PVP-VA 64 (n=2).

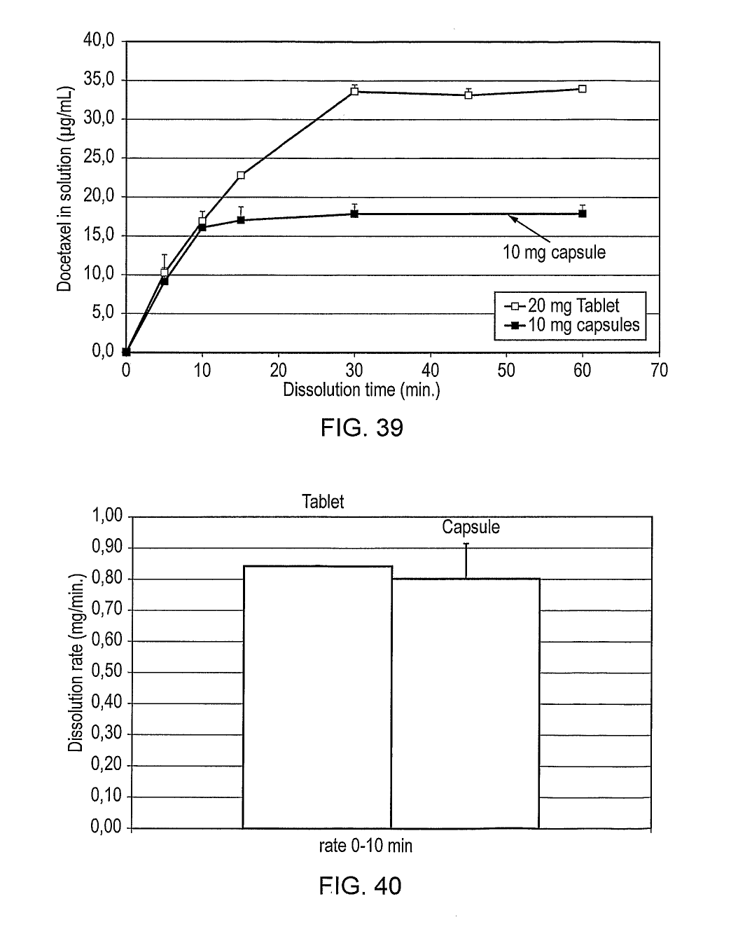

FIG. 39 shows the average dissolution curves of 20 mg docetaxel tablets (n=2) and 10 mg docetaxel capsules (n=6).

FIG. 40 shows the average release rate of docetaxel between 0 and 10 minutes of 20 mg docetaxel tablets (n=2) and 10 mg docetaxel capsules (n=6).

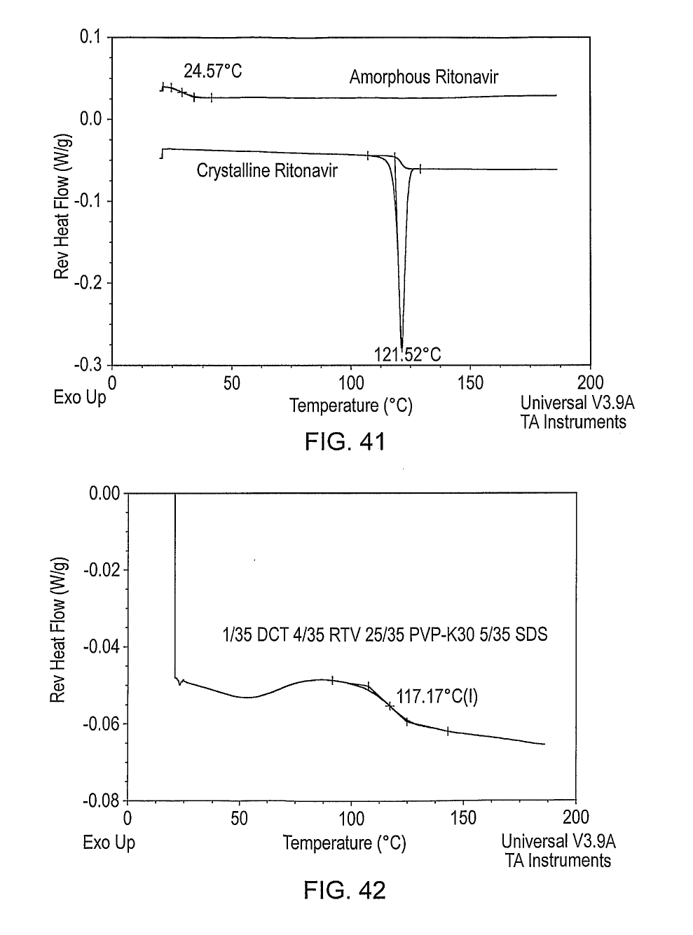

FIG. 41 shows a DSC thermogram of amorphous (spray dried) and crystalline ritonavir.

FIG. 42 shows a DSC thermogram of spray dried solid dispersion powder of the combination of docetaxel, ritonavir, PVP-K30 and SDS.

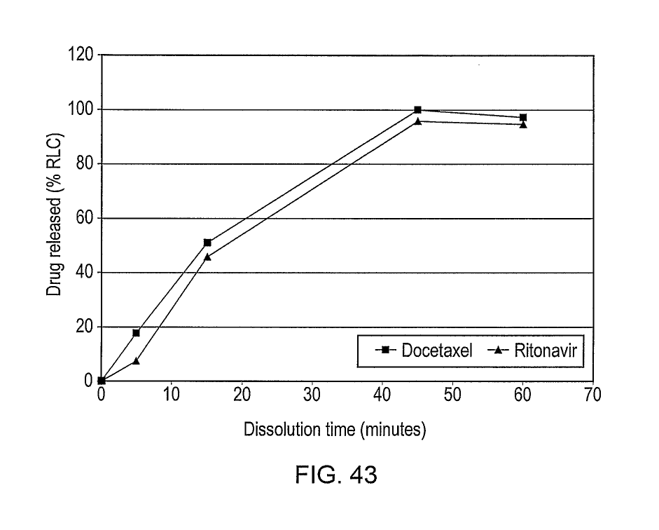

FIG. 43 shows the dissolution profile of docetaxel/ritonavir/PVP-K30/SDS capsules in 1000 mL 0.1 N HCl at 37° C. and 50 RPM.

### EXAMPLE 1

### Oral Formulations of Paclitaxel

### 1.1: Solid Dispersion Versus Physical Mixture

In this experiment the solubility and dissolution rate of a composition comprising a solid dispersion of paclitaxel and PVP-K17 mixed with SDS was compared to a physical mixture of anhydrous paclitaxel, PVP-K17 and SDS.

### 5 mg Capsules of Paclitaxel Solid Dispersions in PVP-K17

A solid dispersion of 20% paclitaxel in PVP-K17 was prepared by dissolving 100 mg of paclitaxel in 10 mL t-butanol and 400 mg PVP-K17 in 6.67 mL water. The paclitaxel/t-butanol solution was added to the PVP-K17/water solution under constant stirring. The final mixture was transferred to 8 mL vials with a maximum fill level of 2 mL. t-butanol and water were subsequently removed by lyophilisation (see table 1 for conditions). 25 mg of a paclitaxel 20%/PVP-K17 solid dispersion (=5 mg paclitaxel) was mixed with 125 mg Lactose, 30 mg sodium dodecyl sulphate, and 30 mg croscarmellose sodium. The resulting powder mixture was encapsulated (see table 2).

TABLE 1
lyophilisation conditions: Lyovac GT4 (AMSCO/Finn-Aqua)

| | | Shelve | Room | Maximum |
|---|---|---|---|---|
| | Time | temperature | pressure | pressure |
| Step | (hh:mm) | (° C.) | (mbar) | (mbar) |
| 1 | 00:00 | Ambient | 1000 | 1000 |
| 2 | 01:00 | −35 | 1000 | 1000 |
| 3 | 03:00 | −35 | 1000 | 1000 |
| 4 | 03:01 | −35 | 0.2 | 0.6 |
| 5 | 48:00 | −35 | 0.2 | 0.6 |
| 6 | 63:00 | 25 | 0.2 | 0.6 |
| 7 | 66:00 | 25 | 0.2 | 0.6 |

TABLE 2
formulation of 5 mg paclitaxel/PVP-K17 solid dispersion capsules

| Component | Amount (mg) |
|---|---|
| paclitaxel (inside the solid dispersion) | 5 mg |
| PVP-K17 (inside the solid dispersion) | 20 mg |
| Lactose monohydrate | 125 mg |
| sodium dodecyl sulphate | 30 mg |
| croscarmellose sodium | 30 mg |

5 mg Capsules of Paclitaxel in a Physical Mixture with PVP-K17

A physical mixture was prepared by mixing 5 mg anhydrous paclitaxel with 20 mg PVP, 125 mg lactose, 30 mg sodium dodecyl sulphate, and 30 mg croscarmellose sodium. The resulting powder mixture was encapsulated.

TABLE 3
formulation of 5 mg paclitaxel/PVP-K17 physical mixture capsules

| Component | Amount (mg) |
|---|---|
| paclitaxel | 5 mg |
| PVP-K17 | 20 mg |
| Lactose monohydrate | 125 mg |
| sodium dodecyl sulphate | 30 mg |
| croscarmellose sodium | 30 mg |

### Dissolution Test

Both capsule formulations were tested in 900 mL of Water for Injection maintained at 37° C. in a USP 2 (paddle) dissolution apparatus with a rotation speed of 75 rpm. In the first experiment, one capsule of each formulation was used. In the second experiment, two capsules of each formulation were used. Samples were collected at various timepoints and analyzed by HPLC-UV (see table 4).

TABLE 4
chromatographic conditions

| Column | Apex octyl 150 × 4.6 mm 5 μm | |
|---|---|---|
| Eluens | Methanol/Acetonitrile/0.02M | |
| | Ammoniumacetate 1/4/5 v/v/v | |
| Flow | 1.0 | mL/min |
| Injection volume | 50 | μL |
| Run time | 15 | minutes |
| Detection wavelength | 227 | nm |

### Results and Conclusions

The results are shown in FIG. 1. The amount of paclitaxel dissolved is expressed relative to the label claims 5 and 10 mg). It can clearly be seen that the dissolution of paclitaxel is greatly improved by the incorporation in a solid dispersion with PVP. The maximum amount of paclitaxel dissolved stays below 20% relative to label claim when a physical mixture is used. When a solid dispersion is used, the solubility is about 65% (5 mg paclitaxel) or over 70% (10 mg paclitaxel). For the 10 mg paclitaxel experiment, this corresponds to an absolute solubility of about 8 μg/ml and this is achieved after about 15 minutes. Therefore, the solid dispersion significantly increases the solubility and also provides a rapid dissolution rate, both of which are important for bioavailability.

In a solid solution or solid dispersion, the amorphous state of the carrier enables thorough mixing of the carrier and taxane. The carrier prevents crystallization during storage as well as during dissolution in aqueous media.

### 1.2: Addition of Sodium Dodecyl Sulphate to the Capsule Formulation

In this experiment, the effect on solubility of the presence or absence of the surfactant SDS in the capsule was determined.

### 20% Paclitaxel Solid Dispersion in PVP-K17

A solid dispersion was prepared by dissolving 100 mg of Paclitaxel in 10 mL t-butanol and 400 mg PVP-K17 in 6.67 mL water. The paclitaxel/t-butanol solution was added to the PVP-K17/water solution under constant stirring. The final mixture was transferred to 8 mL vials with a maximum fill level of 2 mL. t-butanol and water were subsequently removed by lyophilisation (see table 1).

5 mg Paclitaxel Capsules without Sodium Dodecyl Sulphate

25 mg of a paclitaxel 20%/PVP-K17 solid dispersion mg paclitaxel) was mixed with 125 mg Lactose and encapsulated (see table 5).

TABLE 5
formulation of 5 mg paclitaxel/PVP-K17 solid dispersion
without sodium dodecyl sulphate capsules

| Component | Amount (mg) |
|---|---|
| paclitaxel (inside the solid dispersion) | 5 mg |
| PVP-K17 (inside the solid dispersion) | 20 mg |
| Lactose monohydrate | 125 mg |

5 mg Paclitaxel Capsules with Sodium Dodecyl Sulphate

25 mg of a paclitaxel 20%/PVP-K17 solid dispersion mg paclitaxel) was mixed with 125 mg Lactose, 30 mg sodium dodecyl sulphate, and 30 mg croscarmellose sodium. The resulting powder mixture was capsulated (see table 6).

TABLE 6
formulation of 5 mg paclitaxel/PVP-K17 solid dispersion
with sodium dodecyl sulphate capsules

| Component | Amount (mg) |
|---|---|
| paclitaxel (inside the solid dispersion) | 5 mg |
| PVP-K17 (inside the solid dispersion) | 20 mg |
| Lactose monohydrate | 125 mg |
| sodium dodecyl sulphate | 30 mg |
| croscarmellose sodium | 30 mg |

### Dissolution Test

Both capsule formulations were tested in 900 mL of Water for Injection maintained at 37° C. in a USP 2 (paddle) dissolution apparatus with a rotation speed of 75 rpm. Samples were collected at various timepoints and analyzed by HPLC-UV (see table 4).

### Results and Conclusions

The results are shown in FIG. 2. The amount of paclitaxel dissolved is expressed relative to the label claim (in this case 5 mg). The porosity of the lyophilized taxane and carrier solid dispersion was high enough to ensure rapid dissolution when in powder form (results not shown). However, when the powder is compressed in capsules, the wettability is dramatically decreased. Therefore, a surfactant is needed to wet the solid dispersion when it is compressed in capsules or tablets.

It can clearly be seen from FIG. 2 that the dissolution of paclitaxel is greatly improved by the addition of the surfactant sodium dodecyl sulphate. Previous experiments had shown that the addition of croscarmellose sodium, more lactose or the use of larger capsules did not result in increased dissolution rates of the capsule formulation. Again, this shows that with a surfactant like SDS maximum dissolution is achieved in about 10-15 minutes.

### 1.3: Addition of Sodium Dodecyl Sulphate to the Solid Dispersion Formulation

In this experiment, the effect on solubility of adding SDS to the solid dispersion was determined.

### Paclitaxel 40% Solid Dispersion in PVP-K17

A solid dispersion was prepared by dissolving 600 mg of Paclitaxel in 60 mL t-butanol and 900 mg PVP-K17 in 40 mL water. The paclitaxel/t-butanol solution was added to the PVP-K17/water solution under constant stirring. The final mixture was transferred to 8 mL vials with a maximum fill level of 2 mL. t-butanol and water were subsequently removed by lyophilisation (see table 1).

### Paclitaxel 40% Solid Dispersion in PVP-K17 and Sodium Dodecyl Sulphate 10%

A solid dispersion was prepared by dissolving 250 mg of Paclitaxel in 25 mL t-Butanol, and 375 mg PVP-K17 and 62.5 mg sodium dodecyl sulphate (SDS) in 16.67 mL water. The paclitaxel/t-butanol solution was added to the PVP-K17/sodium dodecyl sulphate/water solution under constant stirring. The final mixture was transferred to 8 mL vials with a maximum fill level of 2 mL. t-butanol and water were subsequently removed by lyophilisation (see table 1).

### 25 mg Paclitaxel Capsules of Paclitaxel/PVP-K17 Solid Dispersion

62.5 mg of a paclitaxel 40%/PVP-K17 solid dispersion (=25 mg paclitaxel) was mixed with 160 mg lactose, 30 mg sodium dodecyl sulphate and 10 mg croscarmellose sodium. The resulting powder mixture was encapsulated (see table 7).

TABLE 7
formulation of 25 mg paclitaxel/PVP-K17 solid dispersion capsules

| Component | Amount (mg) |
|---|---|
| paclitaxel (inside the solid dispersion) | 25 mg |
| PVP-K17 (inside the solid dispersion) | 37.5 mg |
| Lactose monohydrate | 125 mg |
| sodium dodecyl sulphate | 30 mg |
| croscarmellose sodium | 10 mg |

### 25 mg Paclitaxel Capsules of Paclitaxel/PVP-K17/Sodium Dodecyl Sulphate Solid Dispersion

68.75 mg of a paclitaxel 40%/PVP-K17/sodium dodecyl sulphate 10% solid dispersion (=25 mg paclitaxel) was mixed with 160 mg lactose and 10 mg croscarmellose sodium. The resulting powder mixture was encapsulated (see table 8).

TABLE 8
formulation of 25 mg paclitaxel/PVP-K17 solid dispersion capsules

| Component | Amount (mg) | |
|---|---|---|
| paclitaxel (inside the solid dispersion) | 25 | mg |
| PVP-K17 (inside the solid dispersion) | 37.5 | mg |
| sodium dodecyl sulphate (inside the solid dispersion) | 6.25 | mg |
| Lactose monohydrate | 125 | mg |
| croscarmellose sodium | 10 | mg |

### Dissolution Test

Both capsule formulations were tested in 500 mL of Water for Injection maintained at 37° C. in a USP 2 (paddle) dissolution apparatus. Rotation speed was set at 75 rpm for the capsule with paclitaxel/PVP-K17/sodium dodecyl sulphate solid dispersion and at 100 rpm for the capsule with paclitaxel/PVP-K17 solid dispersion. Samples were collected at various timepoints and analyzed by HPLC-UV (see table 4).

### Results and Conclusions

The results are shown in FIG. 3. The amount of paclitaxel dissolved is expressed relative to the label claim (in this case 25 mg). It can clearly be seen that the dissolution of paclitaxel from capsules with sodium dodecyl sulphate incorporated in the solid dispersion is comparable to the dissolution of paclitaxel from capsules with sodium dodecyl sulphate added to the capsule. Furthermore only 6.25 mg sodium dodecyl sulphate was used for incorporation into the solid dispersion, while 30 mg sodium dodecyl sulphate was used as addition to the capsule formulation. This shows that less surfactant is required when it is incorporated into the solid dispersion rather than into the capsule in order to achieve the similar results. Another surprising result from this experiment is that both compositions provide an absolute paclitaxel solubility of about 26 μg/ml and this level is reached in 20-30 minutes. This result provides a higher solubility and faster dissolution rate than has previously been achieved.

### 1.4: Influence of Carrier

The solid dispersions used in the experiments of example 1.4 were produced after initial experiments did not show clear differences between drugloads. The 40% drugload was selected because these formulations performed equally to 20% drugload formulation in the afore mentioned experiments and offered the possibility to deliver more taxane in one tablet or capsule.

### Paclitaxel 40% Solid Dispersion in PVP-K12

A solid dispersion was prepared by dissolving 250 mg of paclitaxel in 25 mL t-butanol and 375 mg PVP-K12 in 16.67 mL water. The paclitaxel/t-butanol solution was added to the PVP-K12 water solution under constant stirring. The final mixture was transferred to 8 mL vials with a maximum fill level of 2 mL. t-butanol and water were subsequently removed by lyophilisation (see table 1).

### Paclitaxel 40% Solid Dispersion in PVP-K17

A solid dispersion was prepared by dissolving 600 mg of paclitaxel in 60 mL t-butanol and 900 mg PVP-K17 in 40 mL water. The paclitaxel/t-butanol solution was added to the PVP-K17 water solution under constant stirring. The final mixture was transferred to 8 mL vials with a maximum fill level of 2 mL. t-butanol and water were subsequently removed by lyophilisation (see table I).

### Paclitaxel 40% Solid Dispersion in PVP-K30

A solid dispersion was prepared by dissolving 250 mg of paclitaxel in 25 mL t-Butanol and 375 mg PVP-K30 in 16.67 mL water. The paclitaxel/t-butanol solution was added to the PVP-K30 water solution under constant stirring. The final mixture was transferred to 8 mL vials with a maximum fill level of 2 mL. t-butanol and water were subsequently removed by lyophilisation (see table 1).

### Paclitaxel 40% Solid Dispersion in HP-Cyclodextrin

A solid dispersion was prepared by dissolving 250 mg of paclitaxel in 25 mL t-butanol and 375 mg HP-cyclodextrin in 16.67 mL water. The paclitaxel/t-butanol solution was added to the HP-cyclodextrin water solution under constant stirring. The final mixture was transferred to 8 mL vials with a maximum fill level of 2 mL. t-butanol and water were subsequently removed by lyophilisation (see table 1),

### 25 mg Paclitaxel Solid Dispersion Capsules

62.5 mg of the paclitaxel/carrier solid dispersion (=25 mg paclitaxel) was mixed with 160 mg Lactose, 30 mg sodium dodecyl sulphate and 10 mg croscarmellose sodium. The resulting powder mixture was encapsulated (see table 9).

TABLE 9
formulation of 25 mg paclitaxel/carrier solid dispersion capsules

| Component | Amount (mg) |
|---|---|
| paclitaxel (inside the solid dispersion) | 25 mg |
| carrier (inside the solid dispersion) | 37.5 mg |
| Lactose monohydrate | 125 mg |
| sodium dodecyl sulphate | 30 mg |
| croscarmellose sodium | 10 mg |

### Dissolution Test

All capsule formulations were tested in 500 mL of Water for Injection maintained at 37° C. in a USP 2 (paddle) dissolution apparatus with a rotation speed of 100 rpm. Samples were collected at various timepoints and analyzed by HPLC-UV (see table 4).

### Results and Conclusions

The average results of 2 to 3 experiments are shown in FIG. 4. The amount of paclitaxel dissolved is expressed relative to the label claim (in this case 25 mg). It can clearly be seen that the dissolution of paclitaxel from the PVP-K30 solid dispersion is as fast as the dissolution of paclitaxel from the PVP-K17 solid dispersion. However, the amount of paclitaxel dissolved remains higher throughout the 4 hour experiment in the case of the PVP-K30 solid dispersion.

The chain length of the polymeric carrier determines the time to crystallization in aqueous environments.

### 1.5: Influence of Drug/Carrier Ratio

The solid dispersions used in the experiments of example 1.5 were produced after initial experiments did not show clear differences between carriers. These initial experiments were done before the more detailed experiments of Example 1.4. As a result, PVP-K17 was arbitrarily chosen as carrier for further experiments.

### Paclitaxel 10% Solid Dispersion in PVP-K17

A solid dispersion was prepared by dissolving 100 mg of paclitaxel in 10 mL t-butanol and 900 mg PVP-K17 in 40 mL water. The paclitaxel/t-butanol solution was added to the PVP-K17 water solution under constant stirring. The final mixture was transferred to 8 mL vials with a maximum fill level of 2 mL. t-butanol and water were subsequently removed by lyophilisation (see table 1).

### Paclitaxel 25% Solid Dispersion in PVP-K17

A solid dispersion was prepared by dissolving 250 mg of paclitaxel in 25 mL t-butanol and 750 mg PVP-K17 in 16.67 mL water. The paclitaxel/t-butanol solution was added to the PVP-K17 water solution under constant stirring. The final mixture was transferred to 8 mL vials with a maximum fill level of 2 mL. t-butanol and water were subsequently removed by lyophilisation (see table 1).

### Paclitaxel 40% Solid Dispersion in PVP-K17

A solid dispersion was prepared by dissolving 600 mg of paclitaxel in 60 mL t-butanol and 900 mg PVP-K17 in 6.67 mL water. The paclitaxel/t-butanol solution was added to the PVP-K17 water solution under constant stirring. The final mixture was transferred to 8 mL vials with a maximum fill level of 2 mL. t-butanol and water were subsequently removed by lyophilisation (see table 1).

### Paclitaxel 75% Solid Dispersion in PVP-K17

A solid dispersion was prepared by dissolving 250 mg of paclitaxel in 25 mL t-butanol and 83 mg PVP-K17 in 16.67 mL water. The paclitaxel/t-butanol solution was added to the PVP-K17 water solution under constant stirring. The final mixture was transferred to 8 mL vials with a maximum fill level of 2 mL. t-butanol and water were subsequently removed by lyophilisation (see table 1).

### Paclitaxel 100% Solid Dispersion

A solid dispersion was prepared by dissolving 250 mg of paclitaxel in 25 mL t-butanol. The paclitaxel/t-butanol solution was added to 16.67 mL water under constant stirring. The final mixture was transferred to 8 mL vials with a maximum fill level of 2 mL. t-butanol and water were subsequently removed by lyophilisation (see table 1).

### Dissolution Test

An amount of solid dispersion powder, equal to approximately 4 mg Paclitaxel, was placed in a 50 mL beaker. A magnetic stirring bar and 25 mL water was added to the beaker. The solution was stirred at 720 rpm. Samples were collected at various timepoints and analyzed by HPLC-UV (see table 4).

### Results and Conclusions

The average results of 2 to 3 experiments are shown in FIG. 5. The amount of paclitaxel (PCT) dissolved is expressed relative to the label claim (in this case approximately 4 mg). The influence of the drug/carrier ratio is immediately apparent from FIG. 5. The value of the peak concentration of paclitaxel inversely related to the drug/carrier ratio. The highest peak concentration is reached with the lowest drug/carrier ratio (10%), while the lowest peak concentration is reached with the highest drug/carrier ratio (100%). Furthermore, the AUC-values of the 10% drug/carrier ratio solid dispersion are the highest, followed by the AUC-values of 25, 40, 75 and 100% drug/carrier ratio solid dispersions.

The amount of carrier relative to the amount of drug determines the time to crystallization in aqueous environments.

### 1.6: Presence of Enteric Coating

### Paclitaxel 40% Solid Dispersion in PVP-K17 and Sodium Dodecyl Sulphate 10%

A solid dispersion was prepared by dissolving 250 mg of paclitaxel in 25 mL t-butanol, and 375 mg PVP-K17 and 62.5 mg sodium dodecyl sulphate (SDS) in 16.67 mL water. The paclitaxel/t-butanol solution was added to the PVP-K17/sodium dodecyl sulphate/water solution under constant stirring. The final mixture was transferred to 8 mL vials with a maximum fill level of 2 mL. t-butanol and water were subsequently removed by lyophilisation (see table 1).

### 25 mg Paclitaxel Capsules of Paclitaxel/PVP-K17/Sodium Dodecyl Sulphate Solid Dispersion

68.75 mg of a paclitaxel 20%/PVP-K17/sodium dodecyl sulphate 10% solid dispersion (=25 mg paclitaxel) was mixed with 160 mg lactose and 10 mg croscarmellose sodium. The resulting powder mixture was encapsulated (see table 10).

TABLE 10
formulation of 25 mg paclitaxel/PVP-
K17/SDS solid dispersion capsules

| Component | Amount (mg) | |
|---|---|---|
| paclitaxel (inside the solid dispersion) | 25 | mg |
| PVP-K17 (inside the solid dispersion) | 37.5 | mg |
| sodium dodecyl sulphate (inside the solid dispersion) | 6.25 | mg |
| Lactose monohydrate | 125 | mg |
| croscarmellose sodium | 10 | mg |

### Dissolution Test

The capsules were in duplo subjected to two different dissolution tests. The first test was a two tiered dissolution test, consisting of two hours of dissolution testing in 500 mL simulated gastric fluid without pepsin (SGFₛₚ; see table 11) followed by two hours of dissolution testing in 629 mL simulated intestinal fluid without pepsin (SIFₛₚ; see table 11). The second test was conducted in 500 mL fasted state simulated intestinal fluid (FaSSIF; see table 12) medium for four hours.

Both dissolution tests were performed in a USP 2 (paddle) dissolution apparatus with 500 mL medium maintained at 37° C. and paddle rotation speed 75 rpm. The SGFsp medium was changed to SIFsp medium by addition of 129 mL switch medium. Samples were collected at various timepoints and analyzed by HPLC-UV (see table 4).

TABLE 11
SGFₛₚ, SIFₛₚ and switch medium [14]

| Medium | Volume | Components |
|---|---|---|
| SGFₛₚ | 500 mL | 1.0 g NaCL, 3.5 mL HCl, q.s. 500 mL |
| (USP 26) | | Water for Injection |
| Switch medium | 129 mL | 4.08 g KH₂PO4, 30 mL NaOH solution |
| | | 80 g/L (2.0M), |
| | | 129 mL Water for Injection |
| SIFₛₚ + NaCL | 629 ML | 500 mL SGFₛₚ and 129 mL switch medium |
| (USP 24) | | |

TABLE 12
Fasted state simulated intestinal fluid (FaSSIF) medium [15]

| Component | Amount | |
|---|---|---|
| KH₂PO4 | 3.9 | g |
| NaOH | q.s. pH 6.5 | |
| Na taurocholate | 3 | mM |
| Lecithin | 0.75 | mM |
| KCl | 7.7 | g |

| | |
|---|---|
| Distilled water | q.s. 1 L |

### Results and Conclusions

The results are shown in FIG. 6. The amount of paclitaxel dissolved is expressed relative to the label claim (in this case 25 mg). Paclitaxel dissolution in Fasted state simulated intestinal fluid is approximately 20% higher than in simulated gastric fluid (SGFsp). After two hours in SGFsp the amount of paclitaxel in solution is only slightly increased when the medium is changed to simulated intestinal fluid (SIFsp).

An enteric coating will prevent release of the taxane in the stomach, thereby preventing degradation of the active components. Furthermore, it will enable targeted delivery to the intestines where the taxane is absorbed, thus ensuring that the limited time the taxane is present in solution (before crystallization takes place), is only spent at sites where absorption is possible.

### EXAMPLE 2

### Oral Formulations of Docetaxel

### Materials and Methods

The formulations used in the following experiments were prepared according to the procedures outline below and the compositions depicted in table 13.

### Pure Anhydrous Docetaxel

Anhydrous docetaxel was used as obtained from ScinoPharm, Taiwan.

### Pure Amorphous Docetaxel

Docetaxel was amorphized by dissolving 300 mg of Docetaxel anhydrate in 30 mL of t-butanol. The docetaxel/t-butanol solution was added to 20 mL of Water for Injection (WfI) under constant stirring. The final mixture was transferred to a stainless steel lyophilisation box (Gastronorm size 1/9), t-butanol and water were subsequently removed by lyophilisation (see table 14).

### Physical Mixtures

Physical mixtures were prepared by mixing 150 mg of docetaxel and corresponding amounts of carrier and surfactant (see table 13) with mortar and pestle.

### Solid Dispersions

Solid dispersions were obtained by dissolving 300 mg docetaxel anhydrate in 30 mL of t-butanol, and corresponding amounts of carrier and surfactant (see table 13) in 20 mL of Water for Injection. The docetaxel/t-butanol solution was added to the carrier/surfactant/WfI solution under constant stirring. The final mixture was transferred to a stainless steel lyophilisation box (Gastronorm size 1/9), t-butanol and water were subsequently removed by lyophilisation (see table 14).

TABLE 13
Composition of the tested formulations

| | | | | Amount | | | Amount | | Amount | |
|---|---|---|---|---|---|---|---|---|---|---|
| Formulation | Type | Drug | Part | (mg) | Carrier | Part | (mg) | Surfactant | (mg) | Part |
| A | Pure drug | Anhydrous Docetaxel | 1 | 150 | — | — | | — | | — |
| B | Pure drug | Amorphous Docetaxel | 1 | 450 | — | — | | — | | — |
| C | Physical mixture | Anhydrous Docetaxel | 1/11 | 150 | PVP-K30 | 9/11 | 1350 | SDS | 150 | 1/11 |
| D | Physical mixture | Amorphous Docetaxel | 1/11 | 150 | PVP-K30 | 9/11 | 1350 | SDS | 150 | 1/11 |
| E | Solid dispersion | Amorphous Docetaxel | 1/11 | 300 | PVP-K30 | 9/11 | 2700 | SDS | 300 | 1/11 |
| F | Solid dispersion | Amorphous Docetaxel | 1/11 | 300 | HPβ-CD¹ | 9/11 | 2700 | SDS | 300 | 1/11 |
| G | Solid dispersion | Amorphous Docetaxel | 1/11 | 300 | PVP-K12 | 9/11 | 2700 | SDS | 300 | 1/11 |
| H | Solid dispersion | Amorphous Docetaxel | 1/11 | 300 | PVP-K17 | 9/11 | 2700 | SDS | 300 | 1/11 |
| I | Solid dispersion | Amorphous Docetaxel | 1/11 | 300 | PVP-K25 | 9/11 | 2700 | SDS | 300 | 1/11 |
| J | Solid dispersion | Amorphous Docetaxel | 1/11 | 300 | PVP-K90 | 9/11 | 2700 | SDS | 300 | 1/11 |
| K | Solid dispersion | Amorphous Docetaxel | 5/7 | 300 | PVP-K30 | 5/21 | 100 | SDS | 20 | 1/21 |
| L | Solid dispersion | Amorphous Docetaxel | 1/3 | 300 | PVP-K30 | 1/2 | 450 | SDS | 150 | 1/6 |
| M | Solid dispersion | Amorphous Docetaxel | 1/6 | 300 | PVP-K30 | 2/3 | 1200 | SDS | 300 | 1/6 |
| N | Solid dispersion | Amorphous Docetaxel | 1/21 | 300 | PVP-K30 | 19/21 | 5700 | SDS | 300 | 1/21 |

¹HPβ-CD is hydroxypropyl-β-cyclodextrin

TABLE 14
lyophilisation conditions

| | | Shelve | Room | Maximum |
|---|---|---|---|---|
| | Time | temperature | pressure | pressure |
| Step | (hh:mm) | (° C.) | (mbar) | (mbar) |
| 1 | 00:00 | Ambient | 1000 | 1000 |
| 2 | 01:00 | −35 | 1000 | 1000 |
| 3 | 03:00 | −35 | 1000 | 1000 |
| 4 | 03:01 | −35 | 0.2 | 0.6 |
| 5 | 48:00 | −35 | 0.2 | 0.6 |
| 6 | 63:00 | 25 | 0.2 | 0.6 |
| 7 | 66:00 | 25 | 0.2 | 0.6 |

### Dissolution Test

An amount of powder, equal to approximately 6 mg Docetaxel, was placed in a 50 mL beaker. A magnetic stirring bar and 25 mL water were added to the beaker. The solution was stirred at 720 rpm, and kept at approximately 37° C. Samples were collected at various timepoints, and filtrated using a 0.45 μm filter before they were diluted with a 1:4 v/v mixture of methanol and acetonitrile. The filtrated and diluted samples were subsequently analyzed by HPLC-UV (see table 15).

TABLE 15
chromatographic conditions

| Column | Apex octyl 150 × 4.6 mm 5 μm | |
|---|---|---|
| Eluens | Methanol/Acetonitrile/0.02M | |
| | Ammoniumacetate 1/4/5 v/v/v | |
| Flow | 1.0 | mL/min |
| Injection volume | 10 | μL |
| Run time | 20 | minutes |
| Detection wavelength | 227 | nm |

### 2.1: Formulation Type

In the first experiment, the influence of the formulation type on the solubility of docetaxel was examined. Data from the dissolution test performed on formulations A to E were compared. The results are shown in FIG. 7. Formulation E was tested in quadruplicate, formulation A to D were tested in duplicate.

### Results

Formulation A (pure docetaxel anhydrate) reaches a maximum concentration of approximately 12 μg/mL (4.7% total docetaxel present) after 5 minutes of stirring and reaches an equilibrium concentration of approximately 6 μg/mL (2%) after 15 minutes of stirring.

Formulation B (pure amorphous docetaxel) reaches a maximum of 32 μg/mL (13%) after 0.5 minutes, from 10 to 60 minutes the solubility is comparable to formulation A.

Formulation C (physical mixture of anhydrous docetaxel, PVP-K30 and SDS) reaches a concentration of approximately 85 μg/mL (37%) after 5 minutes. Between 15 and 25 minutes, the docetaxel concentration sharply declines from 85 μg/mL (37%) to 30 μg/mL (12%), after which it further declines to 20 μg/mL (9%) at 60 minutes.

Formulation D (physical mixture of amorphous docetaxel, PVP-K30 and SDS) reaches a maximum docetaxel concentration of 172 μg/mL (70%) after 7.5 minutes. Between 7.5 and 20 minutes, the amount of docetaxel in solution drops to 24 μg/mL (10%). At 60 minutes, the equilibrium concentration of 19 μg/mL (7%) is reached.

Formulation E (solid dispersion of amorphous docetaxel, PVP-K30 and SDS) has the highest maximum concentration of 213 μg/mL (90%) which is reached after 5 minutes. Between 10 and 25 minutes, the amount of docetaxel in solution rapidly declines resulting in an equilibrium concentration of 20 μg/mL (8%) after 45 minutes.

### Conclusions

All formulations initially show a higher solubility, which decreases to an equilibrium solubility after 45 to 60 minutes of stirring. The decrease in solubility is caused by the crystallization of docetaxel as a result of the supersaturated solution. The degree of supersaturation is dependent on the physical state of the drug, i.e. whether it is amorphous or crystalline. When PVP-K30 is the carrier, the supersaturated state is maintained for longer so that the solubility of the docetaxel does not decrease as quickly. Further, the results show that using amorphous docetaxel significantly increases the solubility of docetaxel compared to anhydrous crystalline docetaxel. Further, amorphous docetaxel shows a relatively high dissolution rate, peaking at about 5 to 7.5 minutes.

This experiment shows that the amount of docetaxel in solution is markedly increased by physical mixing of anhydrous docetaxel with PVP-K30 and SDS, and even more by physical mixing of amorphous docetaxel with PVP-K30 and SDS. The biggest increase in solubility, however, is achieved by incorporation of docetaxel in a solid dispersion of PVP-K30 and SDS.

### 2.2: Carrier Type

In the second experiment, the influence of the carrier type on the solubility of docetaxel was examined. Data from the dissolution test performed on formulation E and F were compared. The results are shown in FIG. 8. Formulation E was tested in quadruplicate, formulation F was tested in duplicate.

### Results

Formulation E (solid dispersion of amorphous docetaxel, PVP-K30 and SDS) has a highest maximum concentration of 213 μg/mL (90% of total docetaxel present) which is reached after 5 minutes. Between 10 and 25 minutes, the amount of docetaxel in solution rapidly declines, resulting in an equilibrium concentration of 20 μg/mL (8%) after 45 minutes.

Formulation F (solid dispersion of amorphous docetaxel, HPβ-CD and SDS) reaches a maximum docetaxel concentration of approximately 200 μg/mL (81%) after about 2 minutes. Between 5 and 10 minutes, the amount of docetaxel in solution drops to a value of 16 μg/mL (6%) and after 45 minutes, an equilibrium concentration of 11 μg/mL (4%) is reached.

### Conclusions

This experiment shows that both PVP-K30 and HPβ-CD increase the solubility of docetaxel. When PVP-K30 is used as the carrier compared to HPβ-CD, the maximum docetaxel concentration is slightly higher and the state of supersaturation is maintained longer so that the solubility of docetaxel does not decrease as quickly with time. Further, the equilibrium concentration reached after precipitation of docetaxel is higher with PVP-K30 compared to HPβ-CD.

### 2.3: Chain Length

In the third experiment, the influence of the PVP chain length on the solubility of docetaxel was examined. Data of the dissolution test performed on formulation E and G to J were compared. The results are shown in FIG. 9. Formulation E was tested in quadruplicate, formulation G to J were tested in duplicate.

### Results

Formulation G (PVP-K12) reaches a maximum docetaxel concentration of 206 μg/mL (77% of the total docetaxel present) after 5 minutes. Between 5 and 30 minutes, the amount of docetaxel in solution decreases to 20 μg/mL (7%) and at 45 minutes, the docetaxel concentration is 17 μg/mL (6%).

Formulation H (PVP-K17) reaches a maximum docetaxel concentration of 200 μg/mL (83%) after 5 minutes and maintains this concentration up to 10 minutes of stirring, after which the amount of docetaxel in solution rapidly drops to 44 μg/mL (18%) at 15 minutes and 22 μg/mL (9%) at 30 minutes. The equilibrium concentration between 45 and 60 minutes is approximately 21 μg/mL (8%).

Formulation I (PVP-K25) reaches a maximum docetaxel concentration of 214 μg/mL (88%) after 5 minutes of stirring. The amount of docetaxel in solution decreases between 10 and 30 minutes to 22 μg/mL (9%) and at 60 minutes, the concentration of docetaxel is 19 μg/mL (8%).

Formulation E (PVP-K30) has a maximum docetaxel concentration of 213 μg/mL (90%) which is reached after 5 minutes. Between 10 and 25 minutes, the amount of docetaxel in solution rapidly declines, resulting in an equilibrium concentration of 20 μg/mL (8%) after 45 minutes.

Formulation J (PVP-K90) reaches a maximum docetaxel concentration of 214 μg/mL (88%) after 10 minutes of stirring. At 15 minutes, the amount of docetaxel in solution is still 151 μg/mL (61%). After 60 minutes, the docetaxel concentration has declined to 19 μg/mL (7%).

### Conclusions

This experiment shows that the chain length of PVP influences both the degree of supersaturation and the period the supersaturation is maintained. The use of higher PVP chain lengths results in a higher maximum docetaxel concentrations and a longer period of supersaturation, thus, a higher solubility for a longer period of time.

### 2.4: Drug Load

In the fourth experiment, the influence of the drug load on the solubility of docetaxel was examined. Data from the dissolution tests performed on formulations E and K to N were compared. The results are shown in FIG. 10. Formulation E was tested in quadruplicate, formulation K to N were tested in duplicate.

Formulation N (1/21 docetaxel by weight of total composition; 5:95 w/w docetaxel to PVP) reaches a maximum docetaxel concentration of 197 μg/mL (79% of total docetaxel present) after 10 minutes. After 15 minutes, the amount of docetaxel in solution is still 120 μg/mL (48%) and between 15 and 30 minutes, the docetaxel concentration decreases to 24 μg/mL (12%). At 60 minutes the docetaxel concentration is 20 μg/mL (8%).

Formulation E (1/11 docetaxel by weight of total composition; 10:90 w/w docetaxel to PVP) has a maximum concentration of 213 μg/mL (90%) which is reached after 5 minutes. Between 10 and 30 minutes, the amount of docetaxel in solution rapidly declines and reaches an equilibrium concentration of 20 μg/mL (8%) after 45 minutes.

Formulation M (1/6 docetaxel by weight of total composition; 20:80 w/w docetaxel to PVP) has a docetaxel concentration of 196 μg/mL (80%) after 10 minutes of stirring. The amount of docetaxel in solution decreases between 10 and 30 minutes to 25 μg/mL (10%) and at 60 minutes, the concentration of docetaxel is 18 μg/mL (7%).

Formulation L (1/3 docetaxel by weight of total composition; 40:60 w/w docetaxel to PVP) reaches a docetaxel concentration of 176 μg/mL (71%). Between 10 and 15 minutes, the amount of docetaxel in solution rapidly drops to 46 μg/mL (18%) and after 60 minutes, the amount of docetaxel in solution is 18 μg/mL (7%).

Formulation K (5/7 docetaxel by weight of total composition; 75:25 w/w docetaxel to PVP) reaches a maximum docetaxel value of 172 μg/mL (71%) after 5 minutes of stirring. Between 5 and 10 minutes, the docetaxel concentration sharply declines to 42 μg/mL (17%) and after 60 minutes, a docetaxel concentration of 18 μg/mL (7%) is reached.

### Conclusions

This experiment shows that the amount of PVP-K30 relative to the amount of docetaxel used in the solid dispersions influences both the degree of supersaturation and the period the supersaturation is maintained. The use of higher drugloads results in lower maximum docetaxel concentrations and a shorter period of supersaturation, thus, a lower solubility over time.

2.5: Solubility Comparison with a Prior Art Composition

In this experiment, a composition containing a solid dispersion of 15 mg docetaxel, 135 mg PVP-K30 and 15 mg SDS was compared to the literature data of a composition comprising a solid dispersion of 5 mg docetaxel and PVP-K30 as disclosed in Chen et al. [13]. The solubility results were obtained using the dissolution test described in Chen et al. [13] and are shown in FIGS. 11 and 12. A dissolution test was also conducted in Simulated Intestinal Fluid and compared to the literature data of Chen. The results are shown in FIG. 13.

### Results

From FIG. 11, it can be seen that the composition of Chen et al. can dissolve a maximum of about 80% of the 5 mg docetaxel in the composition in 900 ml water. It took over 5 hours to reach this maximum. The docetaxel, PVP-K30 and SDS composition dissolved 100% of the 15 mg docetaxel in about 60 minutes.

In FIG. 12, the absolute concentration of docetaxel is given. The composition of Chen gave a maximum docetaxel concentration of about 4.2 μg/ml after about 5 hours. The docetaxel, PVP-K30 and SDS composition gave a maximum docetaxel concentration of about 16.7 μg/ml after about 60 minutes.

In FIG. 13, the docetaxel capsules reach a solubility of 28 μg/ml (>90% solubility). The solid dispersion described by Chen et al. (docetaxel+PVP K30) reaches a solubility of 4.2 μg/ml (lower than 80% of the 5 mg docetaxel solid dispersion tested for dissolution in 900 ml). The capsule formulation thus reaches a 6.6 fold better solubility with a higher dissolution rate (maximum reached after 30 minutes versus 90-120 minutes by Chen).

### Conclusions

From these results, it can be seen that the docetaxel, PVP-K30 and SDS composition gave a faster dissolution rate and a higher solubility compared to the composition of Chen. For bioavailability, it is important to look at how fast a drug dissolves and what solubility is reached in 0.5 to 1.5 h.

From the results of Chen, a skilled person would not consider that increasing the amount of docetaxel in the composition would increase the absolute solubility of docetaxel. Since the composition of Chen dissolves only 80% of 5 mg docetaxel (i.e. 4 mg) in 900 ml water, you would not expect that increasing the amount of docetaxel to 15 mg would cause any more than 4 mg docetaxel to dissolve. Thus, you would expect a 15 mg docetaxel composition according to Chen to dissolve a maximum of about 27% docetaxel compared to 100% for the docetaxel, PVP-K30 and SDS composition. Therefore, the docetaxel, PVP-K30 and SDS composition provides surprisingly good results compared to Chen.

### 2.6: Dissolution Test in Simulated Intestinal Fluid Sine Pancreatin (SIFsp)

In this experiment, the dissolution of capsules, containing a solid dispersion of docetaxel, PVP-K30 and SDS, was tested in Simulated Intestinal Fluid sine Pancreatin (SIFsp). The capsules contained 15 mg docetaxel according to Formulation E (see table 13). SIFsp was prepared according to USP 28, Capsules containing 15 mg docetaxel were dissolved in 500 mL USP SIFsp at 37° C. with stirring at 75 rpm. The results are shown in FIGS. 14 and 15.

FIGS. 14 and 15 show that nearly 100% of the docetaxel dissolved. This is equivalent to an absolute docetaxel concentration of about 29 μg/ml and is achieved in about 30 minutes. Thus, the composition provides a relatively high solubility in a relatively short period of time.

### 2.7: Stability

It was found that the solid dispersion of docetaxel, PVP-K30 and SDS according to Formulation E (see table 13) and which was used in capsules for clinical trials (see following Example) is stable both chemically (no degradation) and physically (no changes in solubility characteristics) for at least 180 days when stored between 4-8° C.

### EXAMPLE 3

### Clinical Trial Data with Formulations

### Materials and Methods

10 patients participated in an ongoing clinical phase I trial.

These patients were given the following numbers:

301, 302, 303, 304, 305, 306, 307, 308, 309 and 310.

These patients were given medication which consisted of a liquid formulation of docetaxel or a solid composition comprising a solid dispersion of docetaxel, PVP-K30 and SDS (referred to hereinafter as MODRA).

### Liquid Formulation

Docetaxel dose: 30 mg for all patients (with the exception of patient 306 who received 20 mg docetaxel). The 30 mg dose was prepared as follows: 3.0 mL Taxotere® premix for intravenous administration (containing 10 mg docetaxel per ml in polysorbate 80 (25% v/v), ethanol (10% (w/w), and water) was mixed with water to a final volume of 25 mL. This solution was orally ingested by the patient with 100 mL tap water.

### MODRA

Docetaxel dose: 30 mg; 2 capsules with 15 mg docetaxel per capsule were ingested. Formulation E from the previous example (1/11 docetaxel, 9/11 PVP-K30 and 1/11 SDS) was selected for further testing in the clinical trial. A new batch of formulation E was produced by dissolving 1200 mg docetaxel anhydrate in 120 mL of t-butanol, and 10800 mg PVP-K30 and 1200 mg SDS (see table 13) in 80 mL of Water for Injection. The docetaxel/t-butanol solution was added to the PVP-K30/SDS/WfI solution under constant stirring. The final mixture was transferred to a stainless steel lyophilisation box (Gastronorm size 1/3), t-butanol and water were subsequently removed by lyophilisation (see table 14).

A total of 60 gelatine capsules of size 0 were filled with an amount of solid dispersion equivalent to 15 mg docetaxel, an HPLC assay was used to determine the exact amount of docetaxel per mg of solid dispersion. The assay confirmed that the capsules contained 15 mg docetaxel. Patients took the medication orally on an empty stomach in the morning with 100 mL tap water.

### Patient Treatment

Patients 301, 302, 303, 304 and 305 received only liquid formulation.

Patient 306 received 20 mg docetaxel as liquid formulation+ritonavir in the first cycle and in the second cycle the same medication but with extra ritonavir 4 hours after docetaxel ingestion.

Patients 307, 308, 309 and 310 received liquid formulation and/or MODRA. Cycles were administered in a weekly interval.

According to institutional guidelines, for both oral and i.v. docetaxel all patients were treated with oral dexamethason. A dose of 4 mg dexamethason was given 1 hour prior to the study drugs, followed by 4 mg every 12 hours (2 times). One hour prior to docetaxel treatment, patients also received 1 mg granisetron (Kytril®) to prevent nausea and vomiting.

After drug administration, blood samples were collected for pharmacokinetic analyses. A blank sample was taken before dosing. Blood samples were centrifuged, plasma was separated and immediately stored at −20° C. until analyses. Analysis were performed with validated HPLC methods in a GLP (Good Laboratory Practice) certified laboratory [17].

### Results

Table 16 gives an overview of the individual pharmacokinetic results.

| | | | Tlast | Conc | | |
|---|---|---|---|---|---|---|
| ID | Treatment | Cycle | (h) | l last | AUC last | AUC inf |
| 306 | 20 mg docLF | 1 | 48.03 | 0.668 | 242.7 | 256.5 |
| | 1x RTV | | | | | |
| 306 | 20 mg docLF | 2 | 48.02 | 1.34 | 357.2 | 384.5 |
| | 2x RTV | | | | | |
| 301 | 30 mg docLF | 2 | 47.78 | 1.42 | 556.7 | 586.5 |
| 302 | 30 mg docLF | 2 | 8.15 | 141 | 2227.1 | 3028.1 |
| 303 | 30 mg docLF | 2 | 48 | 3.28 | 663.9 | 745.4 |
| 304 | 30 mg docLF | 2 | 47.77 | 2.67 | 723.4 | 761.3 |
| 305 | 30 mg docLF | 2 | 48.07 | 0.498 | 129.8 | 140.5 |
| 307 | 30 mg docLF | 3 | 23.9 | 5.17 | 754.3 | 822.0 |
| 309 | 30 mg docLF | 1 | 24.02 | 14.1 | 2127.5 | 2327.0 |
| 310 | 30 mg docLF | 1 | 24.17 | 6.17 | 758.7 | 836.1 |
| 307 | MODRA 30 mg | 1 | 24.07 | 4.23 | 420.8 | 473.8 |
| 307 | MODRA 30 mg | 2 | 23.97 | 7.05 | 782.1 | 873.6 |
| 308 | MODRA 30 mg | 1 | 23.95 | 10.9 | 645.7 | 879.2 |
| 308 | MODRA 30 mg | 2 | 24.02 | 7.76 | 507.3 | 625.9 |
| 309 | MODRA 30 mg | 2 | 23.8 | 7.09 | 892.2 | 994.1 |
| 310 | MODRA 30 mg | 2 | 23.63 | 8.52 | 650.7 | 760.3 |

docLF: docetaxel liquid formulation
MODRA: docetaxel capsule formulation
Tlast: time at which last sample for measurement docetaxel concentration was taken (in h)
Conc last: docetaxel concentration at Tlast (in ng/mL)
AUC last: AUC calculated until Conc last (ng · h/mL)
AUC inf: AUClast + extrapolation to infinity (ng · h/mL)
Ritonavir dosage is in all cases 100 mg (capsule, Norvir ®)

Patients 301, 302, 303, 304, 305, 307, 309 and 310 received the liquid formulation. The mean, and the 95% confidence interval for the mean of the AUC (extrapolated to infinity) is: 1156 (+348) ng*h/mL. The inter-individual variability is 85%.

Patient 306 received 20 mg docetaxel (as liquid formulation) concomitantly with 100 mg ritonavir in the first cycle and the same combination, one week later, in the second cycle but with 100 mg extra ritonavir 4 hours after ingestion of docetaxel, i.e. two doses of ritonavir were taken, one at t=0 and the second at t=4 h. The pharmacokinetic curves are depicted in FIG. 16.

Patients 307, 308, 309 and 310 received liquid formulation and/or MODRA. The pharmacokinetic curves are depicted in FIG. 17.

FIG. 18 depicts the pharmacokinetic curves of the patients who received the liquid formulation (307, 309 and 310) and all courses (n=6) of the four patients who received MODRA (307, 308, 309 and 310).

The pharmacokinetic results of the liquid formulation versus MODRA, both in combination with 100 mg ritonavir, are summarized below:

### Liquid Formulation (30 mg Docetaxel)

AUC_{inf}(95% confidence interval of the mean): 1156 (808-1504) ng*h/ml
Inter-individual variability: 85% (n=8)

### MODRA (30 mg Docetaxel)

AUC_{inf}(95% confidence interval of the mean): 768 (568-968) ng*h/ml
Inter-individual variability: 29% (n=4)
Intra-individual variability: 33% (n=2)

The average AUC of MODRA was calculated using the 6 curves from four patients. The first dose of MODRA administered to each patient, was used to calculate the inter-individual variability. The intra-individual variability is based on data from patients 307 and 308 who received two doses of MODRA.

### Conclusions

The tested docetaxel Liquid Formulation results in an AUC value that is approximately 1.5 fold higher than the same dose (30 mg) given in the novel capsule formulation (MODRA).

The inter-individual variability of the liquid formulation is high (85%) while the inter-individual variability of the capsule formulation is substantially lower (29%). This is an important feature of the novel capsule formulation and provides a much better predictable docetaxel exposure. Also for safety reasons low inter-individual variability is very much desired in oral chemotherapy regimens.

The intra-individual variability (limited data) is in the same order of magnitude as the inter-individual variability.

A second boosting dose of 100 mg ritonavir ingested 4 hours after docetaxel administration increases the docetaxel AUC 1.5 fold.

Comparison of Oral Capsule Formulations Compared to i.v. Administration

FIG. 19 shows pharmacokinetic curves after i.v. (20 mg docetaxel as a i.v. 1-hour infusion, Taxotere®) (n=5 patients) and oral administration of docetaxel (30 mg docetaxel; MODRA capsules, see above) (n=4 patients; 6 courses). Both i.v. and oral docetaxel administration was combined with administration of 100 mg ritonavir (capsule, Norvir®). According to institutional guidelines, for both oral and i.v. docetaxel, all patients were treated with oral dexamethason. A dose of 4 mg dexamethason was given 1 hour prior to the study drugs, followed by 4 mg every 12 hours (2 times). One hour prior to docetaxel treatment, patients also received 1 mg granisetron (Kytril®) to prevent nausea and vomiting.

The bioavailability of the MODRA capsules was calculated by:

(AUC 30 mg oral/AUC 20 mg iv)×(20/30)×100%=73% (SD 18%).

This shows that the bioavailability of the capsules is relatively high with a low inter-individual variability.

### EXAMPLE 4

### Further Characterisation of Oral Formulations

### Key Findings:

Formulations produced by spray drying are fully amorphous and have a prolonged duration of the supersaturated state upon dissolution testing compared to formulations produced by lyophilization (see section 7 below).

The equilibrium solubility of docetaxel after precipitation upon dissolution testing is significantly increased in formulations with PVP-VA 64 (40 μg/mL) compared to formulations with PVP-K30 (20 μg/mL) and formulations without a carrier (7 μg/mL) (see section 8 below).

### Summary of Conclusions:

Effect of the type of surfactant on the dissolution of docetaxel (section 1):
All tested surfactants (SDS, CPC, polysorbate 80 and sorbitan monooleate) increase the dissolution rate of docetaxel.
The effect of the surfactant on the dissolution rate of docetaxel seems to correlate well with the HLB-value of the surfactant.
SDS and CPC show a similar increase in dissolution rate of docetaxel.

Effect of the amount of SDS on the dissolution of docetaxel (section 2):
A small amount of SDS (1/41) already increases the dissolution rate of docetaxel.
Larger amounts of SDS (1/11) increase the dissolution rate of docetaxel even more.
The effect of surfactants seems to be related to the compaction level of the powders, it is therefore expected that the difference in docetaxel dissolution rate between powders with and without surfactants will even be greater in tablets.

Amorphous nature of docetaxel (section 3):
Lyophilization of docetaxel results in the disappearance of the diffraction peaks in the X-ray powder diffraction spectrum.
Lyophilization of docetaxel results in the disappearance of the endothermic peak near 165° C. and the appearance of a glass transition near 124° C.
The area of the endothermic peak near 165° C. correlates well with the amount of crystalline material in a physical mixture of amorphous and crystalline docetaxel.

Amorphous nature of paclitaxel (section 4):
Lyophilization of paclitaxel results in the disappearance of the diffraction peaks in the X-ray powder diffraction spectrum.
Lyophilization of paclitaxel results in the disappearance of the endothermic peaks near 58, 80 and 163° C., and the appearance of a endothermic peak near 61° C. and a glass transition near 154° C.

Characterization of solid dispersions of docetaxel (section 5):
Lyophilization of a mixture of docetaxel, PVP-K30 and SDS results in the disappearance of the diffraction peaks belonging to docetaxel in the X-ray powder diffraction pattern.
Diffraction peaks of docetaxel are present in a physical mixture of the solid dispersion components.

Characterization of solid dispersions of paclitaxel (section 6):
paclitaxel solid dispersion systems are comparable to docetaxel solid dispersion systems.

Effect of the production method on the characteristics and performance of docetaxel solid dispersion (section 7):
Spray drying of a mixture of docetaxel, PVP-K30 and SDS results in the disappearance of the diffraction peaks belonging to docetaxel and SDS.
Spray drying of a mixture of docetaxel, PVP-K30 and SDS results in the disappearance of the endothermic peaks in the DSC thermogram belonging to docetaxel and SDS.
Spray drying of a mixture of docetaxel, PVP-K30 and SDS results in a longer time to precipitation compared to a lyophilized mixture of docetaxel, PVP-K30 and SDS upon dissolution testing.

Effect of the type of carrier on the dissolution performance of docetaxel solid dispersions (section 8):
The use of a Polyethylene glycol (1500, 6000 or 20000) instead of PVP-K30 results in a shorter time to precipitation and a lower equilibrium solubility of docetaxel upon dissolution testing.
The use of PVP-VA 64 instead of PVP-K30 results in a comparable time to precipitation and a significantly higher equilibrium solubility of docetaxel upon dissolution testing (40 μg/mL vs. 20 μg/mL respectively).

Production of tablets (section 9):
It is feasible to produce tablets with dissolution rates equal or higher than the currently used capsules.
It is feasible to produce tablets with higher contents of docetaxel than the currently used capsules.

### Materials and Methods

### General

The formulations used in the tests were prepared according to the procedures outlined below and the compositions depicted in Table 18, Table 19, Table 20 and Table 17. The tested drugs were paclitaxel and docetaxel

### Crystalline Drug:

Crystalline drug was used as obtained from the supplier.

### Amorphous Drug:

Drugs were amorphized by dissolving 300 mg of drug in 30 mL of t-Butanol. The drug/t-Butanol solution was added to 20 mL of Water for Injection (WfI) under constant stirring. The final mixture was transferred to a stainless steel lyophilization box (Gastronorm size 1/9), t-Butanol and water were subsequently removed by lyophilization (see Table 20)

### Physical Mixtures of Solid Dispersion Components

Physical mixtures were prepared by mixing 150 mg of docetaxel and corresponding amounts of carrier and surfactant (see Table 17) with mortar and pestle.

TABLE 17
Physical mixtures of solid dispersion components

| | Part | | Part | | Part |
|---|---|---|---|---|---|
| Drag | (mg) | Carrier | (mg) | Surfactant | (mg) |
| docetaxel | 1/11 | PVP-K30 | 9/11 | SDS | 1/11 |
| | (300) | | (2700) | | (300) |

### Physical Mixtures of Amorphous and Crystalline Docetaxel

Physical mixtures were prepared by mixing accurately weighed amounts of crystalline and amorphous docetaxel (see Table 18).

TABLE 18
Physical mixtures of crystalline and lyophilized drug

| | Lyophilized | Crystalline | Crystalline |
|---|---|---|---|
| Description | drug (mg)* | drug (mg)** | drug (% w/w) |
| 0% crystalline | 50 | 0 | 100 |
| 5% crystalline | 47.49 | 2.18 | 4.39 |
| 10% crystalline | 46.75 | 4.85 | 9.40 |
| 25% crystalline | 37.38 | 12.78 | 25.48 |
| 50% crystalline | 26.59 | 23.60 | 47.02 |
| 100% crystalline | 0 | 50 | 100 |

*It is assumed that the amount of crystalline material in the lyophilized drug is, or is close to 0% w/w
**It is assumed that the amount of crystalline material in the crystalline drug is, or is close to 100% w/w

### Solid Dispersions (Lyophilized)

Solid dispersions were obtained by dissolving docetaxel in 30 mL of t-Butanol, and corresponding amounts of carrier and surfactant (see Table 19) in 20 mL of Water for Injection. The docetaxel/t-Butanol solution was added to the carrier/surfactant/Wff solution under constant stirring. The final mixture was transferred to a stainless steel lyophilization box (Gastronorm size 1/9), t-Butanol and water were subsequently removed by lyophilization (see Table 20).

TABLE 19
Solid dispersions produced by lyophilization

| No. | Drug | Part (mg) | Carrier | Part (mg) | Surfactant | Part (mg) |
|---|---|---|---|---|---|---|
| 1 | docetaxel | 1/11 (300) | PVP-K30 | 9/11 (2700) | Polysorbate 80 | 1/11 (300) |
| 2 | docetaxel | 1/11 (300) | PVP-K30 | 9/11 (2700) | Sorbitan | 1/11 (300) |
| | | | | | monooleate | |
| 3 | docetaxel | 1/11 (300) | PVP-K30 | 9/11 (2700) | CPC | 1/11 (300) |
| 4 | docetaxel | 1/10 (300) | PVP-K30 | 9/10 (2700) | — | — |
| 5 | docetaxel | 1/11 (300) | PVP-K30 | 9/11 (2700) | SDS | 1/11 (300) |
| 6 | docetaxel | 2/21 (300) | PVP-K30 | 18/21 (2700) | SDS | 1/21 (150) |
| 7 | docetaxel | 4/41 (300) | PVP-K30 | 36/41 (2700) | SDS | 1/41 (75) |
| 8 | docetaxel | 1/11 (300) | PEG 1500 | 9/11 (2700) | SDS | 1/11 (300) |
| 9 | docetaxel | 1/11 (300) | PEG6000 | 9/11 (2700) | SDS | 1/11 (300) |
| 10 | docetaxel | 1/11 (300) | PEG20000 | 9/11 (2700) | SDS | 1/11 (300) |
| 11 | docetaxel | 1/11 (300) | PVP-VA 64 | 9/11 (2700) | SDS | 1/11 (300) |

TABLE 20
lyophilization conditions

| | | Shelve | Room | Maximum |
|---|---|---|---|---|
| | Time | temperature | pressure | pressure |
| Step | (hh:mm) | (° C.) | (mbar) | (mbar) |
| 1 | 00:00 | Ambient | 1000 | 1000 |
| 2 | 01:00 | −35 | 1000 | 1000 |
| 3 | 03:00 | −35 | 1000 | 1000 |
| 4 | 03:01 | −35 | 0.2 | 0.6 |
| 5 | 48:00 | −35 | 0.2 | 0.6 |
| 6 | 63:00 | 25 | 0.2 | 0.6 |
| 7 | 66:00 | 25 | 0.2 | 0.6 |

### Solid Dispersions (Spray Dried)

Solid dispersions were obtained by dissolving docetaxel in 45 mL of ethanol and 5 mL of WfI. After the drug was completely dissolved, PVP-K30 and SDS (see table 21) were added to the drug/ethanol/WfI solution under constant stirring. The final mixture was transferred to a flask and ethanol and water were subsequently removed by spray drying (see Table 22).

TABLE 21
Solid dispersion produced by spray drying

| | Part | | Part | | Part |
|---|---|---|---|---|---|
| Drug | (mg) | Carrier | (mg) | Surfactant | (mg) |
| docetaxel | 1/11 | PVP-K30 | 9/11 | SDS | 1/11 |
| | (300) | | (2700) | | (300) |

TABLE 22
spray drying conditions

| | | | |
|---|---|---|---|
| Inlet temperature: | 80° C. | Aspirator: | 90% |
| Pump: | 25% | Nitrogen: | 35 |

### Capsules (Used in Sections 1 and 2)

Capsules were produced by weighing an amount of lyophilized solid dispersion powder (1/11 docetaxel, 9/11 PVP-K30 and 1/11 SDS) equivalent to 10-15 mg drug. The solid dispersion powder was grinded with mortar and pestle to a fine powder and encapsulated with a manual capsulation apparatus in size 0 hard gelatin capsules. The amount of docetaxel per capsules was estimated after production by subtracting the net capsule weight from the gross capsule weight and multiplying it by the docetaxel ratio of the solid dispersion powder (see Table 19). The contents of the capsules were confirmed by HPLC quality control.

### Clinical Trial Capsules (Used in Section 9)

Clinical trial capsules were produced by weighing an amount of lyophilized solid dispersion powder (1/11 docetaxel, 9/11 PVP-K30 and 1/11 SDS) equivalent to 10 mg drug, 110 mg lactose monohydrate and 1.1 mg colloidal silicon dioxide. All components were mixed with mortar and pestle until a homogeneous mixture was obtained. The mixture was encapsulated with a manual capsulation apparatus in size 0 hard gelatine capsules.

### Tablets (Used in Section 9)

Tablets were produced by weighing an amount of spray dried solid dispersion powder equivalent (1/11 docetaxel, 9/11 PVP-K30 and 1/11 SDS) to 20 mg docetaxel, 110 mg lactose monohydrate and 110 mg crosslinked polyvinylpyrrolidone. All components were mixed with mortar and pestle until a homogeneous mixture was obtained. The mixture was compacted manually on a excentric press equipped with 13 mm flat tooling. Filling volume was fixed at 13.5 mm and the upper pressure was set at 10.5 mm. Tablets were weighed after compaction and the amount of docetaxel was estimated by multiplying the tablet weight by the product of the weight fraction of drug in the solid dispersion powder (see Table 21) and the weight fraction of the solid dispersion powder in the tablet. The contents of the tablets were confirmed by HPLC quality control.

### Dissolution Screening Test

An amount of powder, equal to approximately 6 mg drug, was placed in a 50 mL beaker. A magnetic stirring bar and 25 mL water were added to the beaker. The solution was stirred at 720 rpm, and kept at approximately 37° C. Samples were collected at various timepoints, and filtrated using a 0.45 μm filter before they were diluted with a 1:4 v/v mixture of methanol and acetonitrile. The filtrated and diluted samples were subsequently analyzed by HPLC-UV (see Table 23). The amount of drug dissolved is expressed as concentration in μg/mL.

### Dissolution Test

Capsules or tablets were placed in a type 2 (paddle) dissolution apparatus, filled with 500 mL WfI at 37° C., the rotational speed of the paddle was 75 rpm. Samples were collected at various timepoints, and filtrated using a 0.45 μm filter before they were diluted 1:1 with a 1:4 v/v mixture of methanol and acetonitrile. The filtrated and diluted samples were subsequently analyzed by HPLC-UV (see Table 23). The amount of docetaxel dissolved is either expressed as concentration in μg/mL or as percentage of the label claim (% RLC). The label claim is the estimated amount of drug present in each capsule or tablet after production.

TABLE 23
Chromatographic conditions

| Column | Apex octyl 150 × 4.6 mm 5 μm | |
|---|---|---|
| Eluens | Methanol/Acetonitrile/0.02M | |
| | Ammoniumacetate 1/4/5 v/v/v | |
| Flow | 1.0 | mL/min |
| Injection volume | 10 | μL |
| Run time | 20 | minutes |
| Detection wavelength | 227 | nm |

### Differential Scanning Calorimetric Measurements

DSC measurements were performed on a Q2000 DSC (TA Instruments, New Castle, Del., USA). Temperature scale and heat flow were calibrated with indium. Samples of approximately 10 mg powder were transferred into Tzero Aluminium pans (TA instruments), hermetically closed and placed in the autosampler. The program listed in Table 24 was used for all samples.

TABLE 24
Modulated DSC method

| Step no. | Action |
|---|---|
| 1 | Data storage: off |
| 2 | Equilibrate at 20.00° C. |
| 3 | Modulate +/−1.00° C. every 60 second |
| 4 | Isothermal for 5.00 min. |
| 5 | Data storage: on |
| 6 | Ramp 2.00° C./min to 190.00° |
| 7 | End of method |

### X-Ray Powder Diffraction Measurements

X-ray powder diffraction measurements were performed on a Phlips X'pert pro diffractiometer equipped with an X-celerator. Samples of approximately 0.5 mm thick were placed in a metal sample holder, placed in the diffractiometer and scanned with the settings depicted in Table 25.

TABLE 25
X-ray diffractiometer settings

| | | | |
|---|---|---|---|
| Tension | 40 kV | Current | 50 mA |
| Range | 10-60°2Theta | Step size | 0.020° |
| Scan speed | 0.002°/s | Tube | Ceramic |
| Focus | Long Fine Focus | Anode | Cu |

### EXPERIMENTS

### Section 1: Effect of the Type of Surfactant on the Dissolution of Docetaxel

### Goal

To determine the effect of the surfactant type, five formulations (see Table 19, formulation 1-5) with different surfactants (see Table 26) were prepared by lyophilization (see Table 20). The selection of surfactants was based on the surfactant class and HLB-value. The chosen surfactants represent all three surfactant classes (anionic, cationic and non-ionic) and a broad range of HLB-values (4.3 to 40) Each formulation had the same amount of docetaxel, PVP-K30 and surfactant. From each formulation three capsules were produced without any additives and subjected to a dissolution test.

### Findings

FIG. 20 shows that between 0 and 5 minutes the amount of docetaxel dissolved in the formulation with SDS is below 10% RLC, while the amount of docetaxel dissolved in the formulation with CPC and Polysorbate 80 are above 30% RLC. At this time, approximately 17% RLC of the docetaxel in the sorbitan monooleate formulation is dissolved. However after 10 minutes the differences in the dissolved amount of docetaxel between the SDS (63% RLC), CPC (75% RLC) and polysorbate 80 (68% RLC) formulations are markedly reduced, while the release of docetaxel from the sorbitan monooleate formulation remains considerably lower with 48% RLC. After 15 minutes the amount of docetaxel released from the formulation with SDS is equal to the amount of docetaxel released from the formulation with CPC. The polysorbate 80 and sorbitan monooleate formulation have lower amounts of docetaxel released, 73% RLC and 63% RLC respectively, but these values are still much higher than the release of docetaxel from the solid dispersion system without an surfactant (37% RLC). After 60 minutes the amount of docetaxel released from the CPC and SDS systems are 87% RLC and 89% RLC respectively, the release from the polysorbate 80 and sorbitan monooleate formulations is lower with 77% RLC and 83% RLC respectively.

### Conclusions

All solid dispersion systems with surfactants increase the dissolution of docetaxel compared to the same solid dispersion system without a surfactant. The effect of CPC and SDS on the dissolution of docetaxel is comparable, the initial differences between these two surfactants are probably a result of the variation in dissolution of the capsule shell. The HLB value of the surfactants seems to correlate well with the performance of the solid dispersion formulations.

Furthermore, dissolution tests with capsules produced from paclitaxel solid dispersion systems have shown even greater changes in dissolution rates due to the incorporation of a surfactant (SDS) in the solid dispersion system. This difference in effect is probably related to the difference in solubility of the active components (paclitaxel vs. docetaxel), it is therefore likely that the differences between the various surfactants will be greater for dosage forms produced from paclitaxel containing solid dispersion systems.

TABLE 26
Surfactant properties

| | | Molecular | | | | HLB- |
|---|---|---|---|---|---|---|
| Name | Chemical formula | weight | CMC* | | Type | value** |
| Sodium Dodecyl | NaC₁₂H₂₅SO₄ | 288.38 | 8.2 | mM¹ | Anionic | 40 |
| sulphate (SDS) | | | 0.23% | w/v | | |
| | | | 2.3 | g/L | | |
| Cetylpyridinium | C₂₁H₃₈NCl | 339.87 | 0.92 | mM² | Cationic | 26 |
| chloride (CPC) | | | 0.03% | w/v | | |
| | | | 0.3 | g/L | | |
| Polysorbate 80 | C₆₄H₁₈₈O₂₈ | 1310 | 0.012 | mM | Non-ionic | 15 |
| | | | 0.0016% | w/v | | |
| | | | 0.016 | g/L | | |
| Sorbitan | C₂₄H₄₄O₆ | 429 | 0.016 | mM³ | Non-ionic | 4.3 |
| monooleate | | | 0.00069% | w/v | | |
| | | | 0.0069 | g/L | | |

*amount of surfactant in capsules is approximately 10 mg, or 0.02 g/L in 500 mL Wfl
¹Handbook of Pharmaceutical excipients
²Choi et al. Journal of Hazardous Materials, 2009, 161 (2-3): 1565-1568
³Korhonen et al. Int. J. Pharm. 2004, 269 (1): 227-239
*CMC = Critical Micelle Concentration;
**HLB-value = Hydrophilic Lypophilic Balance value

### Section 2: Effect of the Amount of Surfactant on the Dissolution of Docetaxel

### Goal

To determine the influence of the amount of SDS four solid dispersion powders containing docetaxel (see Table 19, formulation 4-7) were prepared by lyophilization (see Table 20). The amount of SDS varied between the four formulations while the amount of docetaxel and PVP-K30 were kept constant. From each formulation three capsules were produced without any additives and subjected to a dissolution test.

### Findings

FIG. 21 and FIG. 22 show the results of the dissolution tests. Between 0 and 5 minutes the dissolution of docetaxel is limited in all four formulations. The initial slow dissolution is due to the lag time caused by the dissolution of the capsule shell. After the shell is dissolved, the dissolution of the solid dispersion powders can start. The dissolution of docetaxel from the formulation without an surfactant is considerable slower than the dissolution of docetaxel from the formulation with 1/11 SDS.

While the 1/11 SDS formulation reaches an amount of docetaxel dissolved of 90% RLC within 30 minutes, the formulation without SDS reaches only an amount of docetaxel dissolved of 70% RLC (Relative to Label Claim) after 60 minutes. Furthermore the variation in the release rate of docetaxel between capsules of the formulation without an surfactant is much higher than the variation between the capsules of the formulation with 1/11 SDS.

FIGS. 20 and 21 also show the difference between various amounts of SDS. The dissolution of docetaxel is already improved by addition of only 1/41 SDS to the solid dispersion system, however there is no clear difference between the dissolution patterns of 1/41 and 1/21 SDS. An amount of 1/11 SDS results in the best dissolution pattern of docetaxel compared to 1/21 SDS and 1/41 SDS.

### Conclusions

The incorporation of higher amounts of SDS into the solid dispersion system results in a faster dissolution rate. Incorporation of 1/11 w/w SDS results in the fastest dissolution rate.

It is likely that the compaction of solid dispersion powder necessitates the use of a surfactant. Because the production of tablets results in even higher compaction than the production of capsules, incorporation of a surfactant will be even more necessary in solid dispersion tablets.

Incorporation of SDS into the solid dispersion system ensures a homogeneous distribution of the surfactant improving the wettability of the solid dispersion powder after encapsulation and tabletting.

### Section 3: Amorphous Nature of Docetaxel

### Goal

The physical form of docetaxel after lyophilization was investigated by means of differential scanning calorimetry (DSC) (see Table 24) and X-ray powder diffraction (see Table 25) to determine the degree of crystallinity after lyophilization of docetaxel.

### Findings

FIG. 23 shows the effect of lyophilization on the X-ray powder diffraction pattern of docetaxel. Before lyophilization of docetaxel the X-ray diffraction spectrum has numerous diffraction peaks between 10 and 40° 2 Theta, indicating that docetaxel is in a crystalline state. After lyophilization of docetaxel an amorphous halo is present in the X-ray powder diffraction pattern, indicating that docetaxel is in an amorphous form.

FIG. 24 shows the effect of lyophilization on the DSC thermogram of docetaxel. Before lyophilization of docetaxel the DSC thermogram shows a large endothermic peak at 165° C., possibly caused by a rearrangement of the crystal structure of docetaxel. After lyophilization of docetaxel the DSC thermogram has no endothermic peak at 165° C. However, a broad endothermic peak is present around 50° C. which is caused by the evaporation of water and t-butanol. Furthermore a glass transition can be observed at 124° C., indication that docetaxel is in an amorphous state.

FIG. 25 shows X-ray powder diffraction spectra of various mixtures of amorphous (lyophilized) and crystalline docetaxel. A decrease in the crystalline docetaxel content in the mixture results in a decrease in the intensity and number of diffraction peaks in the X-ray powder diffraction spectra. The X-ray diffraction pattern of the mixture with 5% crystalline docetaxel has no diffraction peaks, this indicates that the lowest detectable amount of crystalline docetaxel with X-ray powder diffraction is above 5% w/w (pure drug substance).

FIG. 26 shows DSC thermograms of various mixtures of amorphous (lyophilized) and crystalline docetaxel. A decrease in the crystalline docetaxel content in the mixture results in a decrease in the size of the endothermic peak at 165° C. and an increase in the broad endothermic peak around 50° C. There is a difference in the size of the endothermic peaks at 50 and 165° C. in the thermograms of 0% crystalline docetaxel (pure lyophilized docetaxel) and 5% crystalline docetaxel, indicating that the lowest detectable amount of crystalline docetaxel with DSC is between 0 and 5% w/w crystalline docetaxel (pure drug substance).

FIG. 27 shows a plot of the peak area at 165° C. in the total heat flow thermogram vs. the amount of crystalline material in the physical mixture of crystalline and amorphous docetaxel (see Table 18). The amount of crystalline material in the amorphous and crystalline drug is assumed to be 0 and 100% w/w respectively. The regression line has an determination coefficient of 0.990 and a regression coefficient of 0.995, this confirms that there is a strong correlation between the peak size at 165° C. and the degree of crystallinity.

### Conclusions

Lyophilization of docetaxel results in a reduction of crystallinity to such a degree that X-ray powder diffraction spectra of lyophilized docetaxel do not show diffraction peaks, as well as DSC thermograms do not show the endothermic peak associated with crystal rearrangement. Furthermore in the DSC thermogram a glass transition appears. This all is indicative that after lyophilization docetaxel is in an amorphous state.

The peak area of the endothermic peak associated with crystal rearrangement correlates well with the crystalline docetaxel content in physical mixtures of amorphous and crystalline docetaxel.

### Section 4: Amorphous Nature of Paclitaxel

### Goal

The physical form of paclitaxel after lyophilization was investigated by means of X-ray diffraction and Differential scanning calorimetry to determine the degree of crystallinity of paclitaxel after lyophilization.

### Findings

FIG. 28 shows the effect of lyophilization on the X-ray powder diffraction pattern of paclitaxel. Before lyophilization of paclitaxel the X-ray diffraction spectrum has numerous diffraction peaks between 10 and 40° 2 Theta, indicating that paclitaxel is in a crystalline state. After lyophilization of paclitaxel an amorphous halo is present in the X-ray powder diffraction pattern, indicating that paclitaxel is in an amorphous form.

FIG. 29 shows the effect of lyophilization on the DSC thermogram of paclitaxel. Before lyophilization of paclitaxel the DSC thermogram shows a large endothermic peak at 58, 80 and 163° C. Both the peak at 58 and 80° C. are caused by the loss of water from the crystal lattice. The endothermic peak at 163° C. is possibly caused by a rearrangement of the crystal structure of paclitaxel. After lyophilization of paclitaxel the DSC thermogram has no endothermic peaks at 58, 80 or 163° C., instead a broad endothermic peak at 61° C. and a glass transition at 154° C. can be observed.

### Conclusions

Lyophilization of paclitaxel results in a reduction of crystallinity to such a degree that X-ray powder diffraction spectra of lyophilized paclitaxel do not show diffraction peaks, as well as DSC thermograms do not show the endothermic peak associated with crystal rearrangement. Furthermore in the DSC thermogram a glass transition appears. This all is indicative that after lyophilization paclitaxel is in an amorphous state.

Section 5: Characterization of Solid Dispersions with Docetaxel

### Goal

To characterize the solid dispersion of docetaxel X-ray diffraction measurements and DSC measurements were performed on physical mixtures and solid dispersions of docetaxel, PVP-K30 and SDS.

### Findings

In FIG. 30 the X-ray diffraction patterns of the solid dispersion components docetaxel, PVP-K30 and SDS are shown. Docetaxel is crystalline and has numerous diffraction peaks between 10 and 40° 2 Theta, SDS is crystalline and has sharp diffraction peaks in between 20 and 22° 2 Theta, PVP-K30 is amorphous and has no diffraction peaks.

FIG. 31 shows the DSC thermograms of the solid dispersion components docetaxel, PVP-K30 and SDS. Docetaxel has a large endothermic peak at 165° C., possibly caused by crystal rearrangement. PVP-K30 has a large endothermic peak near 76° C., caused by the evaporation of water, and a glass transition near 162° C. caused by the difference in heat capacity between the glassy and rubbery state.

SDS has a phase transition near 67° C. probably caused by a small amorphous fraction, and a large endothermic region between 80 and 120° C. containing multiple peaks. These peaks are partly caused by melting of the crystalline bulk of SDS and partly caused by unknown non-reversible endothermic events.

FIG. 32 shows the X-ray diffraction spectra of a physical mixture and a solid dispersion system containing 1/11 docetaxel, 9/11 PVP-K30 and 1/11 SDS. The diffraction peaks of docetaxel and SDS appear in the X-ray diffraction spectrum of the physical mixture, while the diffraction peaks of docetaxel do not appear in the X-ray diffraction spectrum of the solid dispersion system.

FIG. 33 shows the DSC thermograms of a physical mixture and a solid dispersion system containing 1/11 docetaxel, 9/11 PVP-K30 and 1/11 SDS. The thermogram of the physical mixture shows an endothermic dip near 100° C. probably caused by SDS; a small endothermic peak near 162° C. probably caused by crystalline docetaxel and an glass transition near 164° C., probably caused by PVP-K30. The thermogram of the solid dispersion system shows an endothermic peak near 113° C. probably caused by SDS; and a glass transition near 155° C. which is probably caused by the combination of amorphous docetaxel and amorphous PVP-K30.

### Conclusions

An X-ray powder diffraction spectrum of the solid dispersion does not show diffraction peaks belonging to docetaxel, while the X-ray powder diffraction spectrum of a physical mixture containing docetaxel, PVP-K30 and SDS shows diffraction peaks belonging to docetaxel. A DSC thermogram of a lyophilized mixture of docetaxel, PVP-K30 and SDS shows a glass transition at 155° C., probably caused by the molecular mixing of amorphous docetaxel and PVP-K30, while a DSC thermogram of a physical mixture containing docetaxel, PVP-K30 and SDS shows a glass transition temperature at 163°, probably caused by PVP-K30. In addition to this the endothermic peak near 162° is only visible in the DSC thermogram of the physical mixture and not in the DSC thermogram of the solid dispersion.

SDS is present in a crystalline state in both the solid dispersion and the physical mixture and causes the diffraction peaks between 20 and 22° 2 Theta in the X-ray diffraction spectra of both the solid dispersion and physical mixture, and the endothermic peaks near 100° C. and 113° C. in the DSC thermograms of the physical mixture and the solid dispersion respectively.

Furthermore, because the lyophilization of docetaxel alone resulted in amorphous docetaxel (see section 3), it is very likely that lyophilization of a mixture containing docetaxel also results in amorphous docetaxel.

### Section 6: Characterization of Solid Dispersions of Paclitaxel

The available data indicates that paclitaxel solid dispersion systems are comparable to docetaxel solid dispersion systems, i.e. paclitaxel is present in an amorphous state after lyophilization while SDS is not.

### Section 7: Effect of the Production Method on the Characteristics and Performance of Docetaxel Solid Dispersion

### Goal

To determine the influence of the production method on the solid dispersion properties, both lyophilization and spray drying were used to produce a solid dispersion system with 1/11 docetaxel, 9/11 PVP-K30 and 1/11 SDS (see Table 19 and Table 21). Both systems were examined by X-ray diffraction, DSC and a dissolution screening test.

### Findings

FIG. 34 shows the X-ray powder diffraction spectra of solid dispersions produced by lyophilization and spray drying. The solid dispersion produced by lyophilisation (freeze drying) is partly crystalline because there are diffraction peaks present in the spectrum between 20 and 22° 2 Theta. These diffraction peaks belong to the SDS (FIG. 30). The solid dispersion produced by spray drying is fully amorphous, as can be concluded from the absence of diffraction peaks in the X-ray powder diffraction pattern.

FIG. 35 shows the DSC thermograms of solid dispersions produced by lyophilization and spray drying. The thermogram of the solid dispersion produced by lyophilization shows an endothermic peak near 113° C. probably caused by SDS; and a glass transition near 155° C., which is probably caused by the combination of amorphous docetaxel and amorphous PVP-K30. The solid dispersion produced by spray drying only shows a glass transition near 147° C., indicative for a fully amorphous system.

FIG. 36 shows the dissolution screening curves of solid dispersions produced by lyophilization and spray drying. The solid dispersion produced by lyophilization reaches the peak docetaxel concentration after 5 minutes and starts to precipitate. The solid dispersion produced by spray drying reaches the peak docetaxel concentration after 10 minutes and starts to precipitate after 15 minutes.

### Conclusions

The use of spray drying compared to lyophilization in the production of solid dispersions of docetaxel lead to a more amorphous system which results in an improved performance in dissolution screening tests in terms of a prolonged supersaturated state.

Furthermore, the powder obtained after spray drying is less static and has a more uniform particle size, making it more suitable for further processing compared to the lyophilized product.

### Section 8: Effect of the Type of Carrier on the Dissolution Performance of Docetaxel Solid Dispersions

### Goal

To test the influence of different carriers on the dissolution performance of docetaxel solid dispersions. Various carriers were used in the production of solid dispersion systems containing 1/11 docetaxel, 9/11 carrier and 1/11 SDS (see Table 19, formulation 8-11). All systems were subjected to a dissolution screening test.

### Findings

FIG. 37 shows the dissolution screening curves of solid dispersion systems containing PVP-K30, PEG 1500, PEG 6000 or PEG20000. All systems reach a comparable docetaxel peak concentration after 5 minutes, however precipitation of docetaxel already starts after 5 minutes for all three PEG systems. Furthermore, the amount of docetaxel in solution after precipitation is approximately 8 μg/mL in the PEG containing solid dispersion systems, while the PVP-K30 containing system reaches an amount of docetaxel in solution after precipitation of 20 μg/mL.

FIG. 38 shows the dissolution screening curves of solid dispersions containing PVP-K30 or PVP-VA 64. For both systems the same docetaxel peak concentration is reached after 5 minutes and for both systems docetaxel starts to precipitate after approximately 10 minutes. There is however a significant difference in the amount of docetaxel in solution after precipitation: PVP-VA 64 40 μg/mL and PVP-K30 20 μg/mL.

### Conclusions

Solid dispersion systems containing PEG perform worse in dissolution screening tests than solid dispersion systems containing PVP-K30.

Solid dispersion systems containing PVP-VA 64 reach significantly higher concentrations of docetaxel after precipitation than solid dispersion systems containing PVP-K30.

Furthermore, because paclitaxel has a lower solubility than docetaxel, the use of PVP-VA 64 might especially be helpful in paclitaxel containing solid dispersion systems.

### Section 9: Production of Tablets

### Goal

To investigate the feasibility of the production of tablets from docetaxel solid dispersions.

### Findings

FIG. 39 shows the average dissolution curves of 20 mg docetaxel tablets and 10 mg docetaxel capsules which are currently used in clinical trials. FIG. 40 shows the average dissolution rates of the tablets and capsules between 0 and 10 minutes. The tablets show a steady dissolution rate between 0 and 30 minutes until a concentration of 35 μg/mL is reached. The capsules show a steady dissolution rate between 0 and 10 minutes until a concentration of approximately 16 μg/mL is reached. Between 0 and 10 minutes the release rate of docetaxel from both capsules and tablets is approximately 0.8 mg/min.

### Conclusion

Production of tablets of docetaxel containing solid dispersion systems is feasible. The dissolution rate of docetaxel tablets is comparable to the dissolution rate of the docetaxel capsules currently used in clinical trials.

### EXAMPLE 5

### Addition of an Additional Pharmaceutically Active Ingredient to the Docetaxel Solid Dispersion

### Production of Amorphous Ritonavir

### Solid Components:

300 mg ritonavir

### Solvents:

45 mL ethanol
5 mL water for injection

Ritonavir was dissolved in the ethanol water mixture and spray dried with a Buchi 290 mini spray dryer (see table 27).

### Production of a Combination Product of Docetaxel, Ritonavir, PVP and SDS

### Solid Components:

1 gram of docetaxel anhydrate
4 grams of ritonavir
25 grams of PVP-K30
5 grams of SDS

### Solvents:

900 ml ethanol

### 100 mL Water for Injection

All solid components were dissolved in the ethanol-water mixture and spray dried with a Buchi 290 mini spray dryer (see table 27). This resulted in a visually homogenous white powder. This solid dispersion powder, equivalent to approximately 12.5 mg docetaxel and 50 mg ritonavir (per capsule), was manually encapsulated in size 0 hard gelatine capsules.

TABLE 27
spray drying conditions

| | | | |
|---|---|---|---|
| Inlet temperature: | 80° C. | Aspirator: | 90% |
| Pump: | 25% | Nitrogen: | 35% |

TABLE 28
Modulated DSC method

| Step no. | Action |
|---|---|
| 1 | Data storage: off |
| 2 | Equilibrate at 20.00° C. |
| 3 | Modulate +/−1.00° C. every 60 second |
| 4 | Isothermal for 5.00 min. |
| 5 | Data storage: on |
| 6 | Ramp 2.00° C./min to 190.00° |
| 7 | End of method |

FIG. 41 shows a DSC thermogram of amorphous (spray dried) and crystalline ritonavir. Amorphous ritonavir exhibits a Tg around 25° C., crystalline exhibits a melting endotherm around 122° C. It is concluded that spray drying results in amorphous ritonavir.

FIG. 42 shows a DSC thermogram of spray dried solid dispersion powder of the combination of docetaxel, ritonavir, PVP-K30 and SDS. The thermogram shows a single Tg around 117° C. and no melting endotherms of ritonavir, docetaxel, PVP-K30 or SDS.

FIG. 43 shows the dissolution profile of docetaxel/ritonavir/PVP-K30/SDS capsules in 1000 mL 0.1 N HCl at 37° C. and 50 RPM. Each capsule contained approximately 12.5 mg docetaxel and approximately 50 mg ritonavir. Both docetaxel and ritonavir are completely released within 45 minutes (n=3).

The foregoing Examples are intended to illustrate specific embodiments of the present invention and are not intended to limit the scope thereof, the scope being defined by the appended claims. All documents cited herein are incorporated herein by reference in their entirety.

### REFERENCES

1. Demario M D, Ratain M J. J Clin Oncol 1998; 16:2557-2567.
2. Liu G, Franssen E, Fitch M I et al. J Clin Oncol 1997; 15:110-115.
3. Meerum Terwogt J M, Beijnen J H, ten Bokkel Huinink W W et al. Lancet 1998; 352:285.
4. Sparreboom A, van Asperen J, Mayer U et al. Proc Natl Acad Sci USA 1997; 94:2031-2035.
5. Kuhn J, Rizzo J, Eckhardt J et al. Proc Am Soc Clin Oncol 1995; 14:474.
6. Schellens J H M, Creemers G J, Beijnen J H et al. Br J Cancer 1996; 73:1268-1271.
7. Hellriegel E T, Bjornnson T D, Hauck W W. Clin Pharmacol Ther 1996; 60:601-607.
8. Huizing M T, Giaccone G, van Warmerdam L J C et al. J Clin Oncol 1997; 15:317-329.
9. Malingré M M, Richel D J, Beijnen J H et al. J Clin Oncol 2001; 19:1160-1166.
10. Mastalerz H, Cook D, Fairchild C R, Hansel S, Johnson W, Kadow J F, Long B H, Rose W C, Tarrant J, Wu M J, Xue M Q, Zhang G, Zoeckler M, Vyas D M. Bioorg Med. Chem. 2003; 11: 4315-23.
11. Serajuddin A T. J Pharm Sci 1999; 88(10):1058 1066.
12. Karanth H, Shenoy V S, Murthy R R. AAPS PharmSciTech 2006; 7(4):87.
13. Chen J, et al. Drug Development and Industrial Pharmacy 2008; 34:588-594.
14. Schellekens R C, Stuurman F E, van der Weert F H, Kosterink J G, Frijlink H W. Eur. J. Pharm. Sci. 2007 January; 30(1): 15-20.
15. Dressman J B, Reppas C. Eur. J. Pharm. Sci. 2000 October; 11: S73-S80.
16. The Handbook of Pharmaceutical Excipients. Fifth Edition. Edited by Raymond Rowe, Paul Sheskey and Sian Owen. See sections on Povidone, PVP VA and PEG.
17. Kuppens I E, Van Maanen M J, Rosing H, Schellens J H M, Beijnen J H. Biomed Chromatogr 2005; 19:355-361.
18. Kawakami (2009) Journal of Pharmaceutical Sciences. 98(9) 2875-2885. Published online in Wiley Interscience (www.interscience.wiley.com) DOI 10.1002/jps.21816

1. A solid pharmaceutical composition for oral administration comprising a substantially amorphous taxane, a hydrophilic carrier and a surfactant, wherein the substantially amorphous taxane is prepared by a solvent evaporation method.

2. The composition of claim 1, wherein the solvent evaporation method is spray drying.

3. The composition of claim 1, wherein the taxane and the carrier are in the form of a solid dispersion.

4. The composition of claim 1, wherein the taxane, the carrier and the surfactant are in the form of a solid dispersion.

5. The composition of claim 3, wherein the solid dispersion is prepared by a solvent evaporation method.

6. The composition of claim 5, wherein the solvent evaporation method is spray drying.

7. (canceled)

8. The composition of claim 7, wherein the taxane is selected from the group consisting of docetaxel, paclitaxel, functional derivatives thereof and pharmaceutically acceptable salts or esters thereof.

9. (canceled)

10. The composition of claim 1, wherein the carrier is selected from PVP, PVP-VA and PEG.

11-14. (canceled)

15. The composition of claim 1, wherein the surfactant is selected from the group consisting of SDS, sorbitan esters (sorbitan fatty acid esters), polyoxyethylene sorbitan fatty acid esters and CPC.

16-17. (canceled)

18. The composition of claim 17, wherein the taxane to carrier weight ratio is between about 0.01:99.99 w/w and about 30:70 w/w.

19. (canceled)

20. The composition of claim 19, wherein the weight ratio of surfactant, to taxane and carrier combined, is between about 2:98 w/w and about 17:83 w/w.

21. The composition of claim 1, further comprising one or more additional pharmaceutically active ingredients.

22. The composition of claim 23, wherein one or more of the additional pharmaceutically active ingredients is a CYP3A4 inhibitor.

23. (canceled)

24. The composition of claim 21, wherein the taxane, the carrier, the surfactant and the one or more additional pharmaceutically active ingredients are in the form of a solid dispersion.

25. The composition of claim 24, wherein the solid dispersion is prepared by a solvent evaporation method.

26. The composition of claim 25, wherein the solvent evaporation method is spray drying.

27-28. (canceled)

29. A method of treating a neoplastic disease, comprising administering to a subject in need of such treatment an effective amount of the composition of claim 1.

30. A method of preparing the composition of claim 1 comprising the steps of: preparing an amorphous taxane using a solvent evaporation method; and combining the amorphous taxane with a hydrophilic carrier and a surfactant to produce the composition.

31. A pharmaceutical composition for oral administration comprising a substantially amorphous taxane and a hydrophilic carrier, wherein the substantially amorphous taxane is prepared by spray drying.

32. (canceled)

33-35. (canceled)

36. A solid pharmaceutical composition for oral administration comprising a substantially amorphous taxane and one or more pharmaceutically acceptable excipients, wherein the substantially amorphous taxane is prepared by spray drying.
